(19) United States
(12) Patent Application Publication
(10) Pub. No.: US 2024/0084027 A1
(43) Pub. Date: Mar. 14, 2024

(54) ANTIBODIES BINDING CD40 AND PD-L1 AND USES THEREOF

(71) Applicant: BEIJING MABWORKS CO., LTD., Beijing (CN)

(72) Inventors: Jiangmei LI, Beijing (CN); Xuechen ZHOU, Beijing (CN); Wenqi HU, Beijing (CN); Feng LI, Beijing (CN)

(21) Appl. No.: 18/449,860

(22) Filed: Aug. 15, 2023

(30) Foreign Application Priority Data
Aug. 26, 2022 (CN) 202211036814.1

(51) Int. Cl. C07K 16/28; A61P 35/00; C07K 16/46

(52) U.S. Cl. CPC C07K 16/2878; A61P 35/00; C07K 16/2827; C07K 16/468; A61K 2039/505; C07K 2317/31; C07K 2317/569; C07K 2317/622; C07K 2317/75; C07K 2317/76

(57) ABSTRACT

Disclosed is a multi-specific antibody targeting CD40 and PD-L1, as well as its use in the treatment of diseases such as tumors.

### INCORPORATION BY REFERENCE

This application claims priority to Chinese Patent Application No. 202211036814.1 filed on Aug. 26, 2022.

The foregoing application, and all documents cited therein or during its prosecution (“appln cited documents”) and all documents cited or referenced herein (including without limitation all literature documents, patents, published patent applications cited herein)(“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

### SEQUENCE STATEMENT

The instant application contains a Sequence Listing XML labeled “55556-00104SequenceListingXML” which was created on Aug. 4, 2023 and is 58 kb. The entire content of the sequence listing is incorporated herein by reference in its entirety.

### FIELD OF THE INVENTION

The present disclosure relates to a multi-specific antibody targeting CD40 and PD-L1 and its use in treating diseases such as tumors.

### BACKGROUND OF THE INVENTION

The immune checkpoint molecules, including inhibitory and stimulatory ones, function to modulate the immune system. The inhibitory immune checkpoint molecules play a key role in preventing the immune system from attacking healthy cells, while the stimulatory immune checkpoint molecules are necessary for initiation of immune responses.

### PD-I and PD-L1

PD-1, an immune checkpoint molecule that exerts inhibitory effect on immune responses, is mainly expressed on memory T cells, and also found on B cells, activated monocytes, dendritic cells and natural killing (NK) cells (Keir ME et al., (2008) Annu Rev Immunol, 26:677-704; Ishida Y et al., (1992) EMBO J. 11(11): 3887-3895). PD-L and PD-L2 are PD-1's ligands. The PD-L1 molecules are constitutively expressed on antigen presenting cells, T cells. B cells, monocytes and epithelial cells, and their levels are upregulated in many cells in the presence of proinflammatory cytokines (Keir ME et al., (2008) supra; Chen J et al., (2016) Ann Oncol. 27(3):409-416). Studies have shown that the PD-L1 signaling may exert inhibitory effects on T cells in several ways, and also on B cell and NK cell-mediated lysis (Dong H et al., (1999) Nat Med. 5(12):1365-1369; Keir ME et al., (2008) supra; Chen J el al., (2016) supra; Terme M et al., (2011) Cancer Res. 71(16):5393-5399; Fanoni D et al., (2011) Immunol Lett. 134(2):157-160).

The PD-1/PD-L1 signaling pathways may be employed by e.g., tumor cells to evade immune surveillance. In particular, the constitutive expression of the PD-L1 molecules is found on many kinds of tumor cells, as well as on myeloid cells such as macrophages and dendritic cells in the tumor microenvironment. The PD-L1-PD-1 interaction in the tumor microenvironment may induce T cell dysfunction and anergy and IL-10 secretion, suppress CD8⁺ T cell-mediated tumor cell death, and promote tumor cell growth (Zou W, Chen L. (2008) Nat Rev Immunol. 8(6):467-477; Sun Z et al., (2015) Cancer Res. 75(8); 1635-1644).

PD-1 or PD-L1 blockade by antibodies may lead to durable tumor regression in many cancer types, including solid tumors and hematological tumors. Currently, there are more than 1000 undergoing clinical trials involving PD-1/PD-L1 blockade in e.g., melanoma, non-small cell lung cancer, renal cell carcinoma, Hodgkin lymphoma, bladder cancer, head and neck cancer, neuroendocrine cancer, solid cancers with microsatellite instability or mismatch repair deficiency, mantle cell lymphoma, diffuse large B-cell lymphoma and follicular lymphoma (Akinleye A, Rasool Z. (2019), J Hematol Oncol, 12(1):92; Chong Sun et al., (2018) Immunity 20; 48(3):434-452). FDA has approved three anti-PD-L1 antibodies for cancer treatment, i.e., Atezolizumab, Durvalumab and Avelumab. The clinical data have revealed atezolizumab's potent efficacy on several solid tumors and hematological tumors, including non-small cell lung cancer, melanoma, renal cell cancer, colorectal cancer, gastric cancer, squamous cell carcinoma of head and neck, and urothelial carcinoma. Similarly, durvalumab, a fully human monoclonal antibody, has shown clinical efficacy on e.g., urothelial carcinoma and non-small cell lung cancer, and avelumab administration has resulted in good clinical outcomes in e.g., Merkel-cell carcinoma, urothelial carcinoma, and triple-negative breast cancer.

In addition, the PD-1/PD-L1 pathway blockade also showed positive results in treating acute or chronic viral, bacterial and parasitic infections in pre-clinical and clinical researches (Jubel J M et al., (2020) Front Immunol, 11:487). For example, the administration of anti-PD-L1 antibodies increased IFN-γ, IL-2 and TNFα levels, enhanced T cell function, and alleviated viremia in chronic infection of e.g., hepatitis B virus (HBV), hepatitis C virus (HCV), human immunodeficiency virus (HIV), or simian immunodeficiency virus (SIV).

### CD40

CD40, also referred to as tumor necrosis factor receptor superfamily member 5 or TNFR5, is a transmembrane costimulatory protein expressed on antigen presenting cells such as B cells, macrophages, and dendritic cells. Binding of this protein with CD40L (CD154), the major ligand expressed primarily by activated T lymphocytes and platelets, activates antigen presenting cells and triggers a variety of downstream signaling pathways, including those relevant to immune cell activation and proliferation, and production of cytokines and chemokines, enhancing cellular and immune functions (Ara A el al., (2018) Immunotargets Ther 7: 55-61).

Several anti-CD40 antibodies have been developed for potential tumor treatment. CP-870,893, a fully human IgG2 anti-CD40 agonistic antibody developed by Pfizer, can activate dendritic cells and has shown clinical efficacy in a number of settings of patients with advanced cancers (Vonderheide et al., (2007) J Clin Oncol 25(7): 876-883; Gladue et al., (2011) Cancer Immunol Immunother 60(7): 1009-1017; Beatty et al., (2013) Expert Rev Anticancer Ther 17(2): 175-186; Vonderheide et al., (2013) Oncoimmunology 2(1): e23033; Nowak et al., Ann Oncol 26(12): 2483-2490; 2015 U.S. Pat. No. 7,338,660). Dacetuzumab, also known as SGN-40, a humanized IgG1 agonistic anti-CD40 antibody developed by Seattle Genetics, has shown anti-tumor activity when given intravenously every week, especially in patients with diffuse large B-cell lymphoma. Preclinical data also showed synergic effect of Dacetuzumab with other agents such as the anti-CD20 mAb rituximab (Lapalombella et al., (2009) Br J Haematol 144(6): 848-855; Hussein et al., (2010) Haematologica 95(5): 845-848; de Vos et al., (2014) J Hematol Oncol 7: 44). Chi Lob7/4, another chimeric IgG1 agonistic anti-human CD40 antibody developed by Cancer Research UK, is undergoing initial clinical testing. Eleven of the 21 patients showed stable disease with no complete or partial responses (Chowdhury et al. (2014) Cancer Immunol Res 2(3): 229-240).

A lot of agonistic anti-CD40 antibodies need to trimerize before binding the CD40 molecule, otherwise the downstream signaling will not be activated. The antibody trimers are formed by cross linking when the antibodies bind their Fc regions to the FecγRllBs on e.g., immune cells. The liver is the main site where the immunocomplexes are cleared, and the high expression levels of FcγRIIB on the liver sinusoidal endothelial cells may likely induce antibody cross linking, such that the immune cells are activated and excessive cytokines are released, leading to hepatotoxicity.

### Bi- or Multi-Specific Antibodies

The antibodies blocking PD-1-PD1-L1 interaction, which relive immunosuppression, as described above, have shown very good efficacy in cancer treatment. However, only a few patients are responsive to and benefit from the anti-PD-1/PD-L1 monotherapies.

Why are patients not sensitive to the therapies targeting PD-1/PD-L1? One important reason may be that to relieve immunosuppression is not sufficient to activate immune cells and induce tumor cell death. Thus, the inventors of the application thought of combining the PD-1/PD-L1 blockade with the immune system-activating agents, e.g., the agonistic anti-CD40 antibodies, which two may synergize to trigger higher immune responses within the tumor microenvironment with e.g., increased tumor-infiltrating immune cells, convening the ‘cold’ tumors to ‘hot’ ones. However, the clinical application of immune-stimulatory antibodies is limited due to their high toxicity. For example, as described above, the agonistic anti-CD40 antibodies are likely to cause hepatotoxicity.

How to block the PD-1-PD-L1 interaction while avoiding the side effects accompanied by the CD40 signaling activation is a problem that needs to be dealt with.

Citation or identification of any document in this application is not an admission that such document is available as prior art to the present invention.

### SUMMARY OF THE INVENTION

The inventors of the application have constructed several bi- or multi-specific anti-CD40/PD-L1 antibodies with optimized structures, which have no FcR binding affinity and can reduce non-specific CD40 signaling activation, such that the antibodies gather around the tumors having high PD-L1 expression levels and cross link through binding to these PD-L1 molecules to initiate CD40 signaling activation.

The bi- or multi-specific antibodies of the disclosure may relieve immunosuppression and activate the immune responses at the same time, enhancing the effectiveness of PD-L1 blockade, inducing higher immune responses within the tumor microenvironment, and reducing hepatotoxicity caused by the agonistic anti-CD40 antibodies. In other words, the bi- or multi-specific antibodies can block PD-L1 signaling and further activate the immune responses through CD40 signaling, and more importantly, they can only cross link and activate CD40 signaling at the sites with high PD-L1 expression levels, resulting in immune response activation specific to tumors and reduced or eliminated toxicity to e.g., livers. With the application of such antibodies, the PD-1/PD-L1 blockade may synergize with CD40 signaling activation, expanding the therapeutic window.

Therefore, in a first aspect, the present application provides a bi- or multi-specific antibody, which may comprise a) a PD-L1 binding domain, and b) a CD40 binding domain.

The PD-L1 binding domain may block PD-1-PD-L1 binding/interaction and antagonize/inhibit the PD-1 signaling pathway. In other words, it does not trigger the PD-1 signaling pathway in the PD-1-expressing cells.

The CD40 binding domain may bind CD40 and activate CD40 signaling. In other words, it may be an agonistic anti-CD40 binding domain.

The bi- or multi-specific antibody of the disclosure may be an IgG like antibody, comprising the CD40 binding domain in the full-length antibody, Fab-Fab or Fv-Fv format, and the PD-L1 binding domain in the single-chain variable fragment (scFv) or nanobody format. Alternatively, the bi- or multi-specific antibody of the disclosure may be an IgG like antibody, comprising the PD-L1 binding domain in the full-length antibody, Fab-Fab or Fv-Fv format, and the CD40 binding domain in the single-chain variable fragment (scFv) or nanobody format.

The inventors of the application compared the structures of various bi- or multi-specific antibodies as prepared, and found that, the antibodies comprising the CD40 binding domain in the full-length antibody, Fab-Fab or Fv-Fv format, and the PD-L1 binding domain linked to the CD40 binding domain in the single-chain variable fragment (scFv) or nanobody format may exhibit superior CD40 binding activity. PD-L1 binding activity, PD-1-PD-L1 blocking activity, CD40 signaling activation capability, T cell activation capability, and/or anti-tumor efficacy. Especially, the PD-L1 binding domain in the scFv or nanobody format may be linked to the C terminus of the heavy chain constant region of the CD40 binding domain in the full-length antibody or Fab-Fab format.

The multi-specific antibody of the disclosure may comprise one CD40 binding domain in the full-length antibody, Fab-Fab or Fv-Fv format, and one or two PD-L1 binding domains in the scFv or nanobody format. Especially, the PD-L1 binding domain in the scFv or nanobody format may be linked to the C terminus of the heavy chain constant region of the CD40 binding domain in the full-length antibody or Fab-Fab format.

Therefore, the bi- or multi-specific antibody of the disclosure may comprise:
i) a first polypeptide chain, comprising, from N terminus to C terminus, an anti-CD40 heavy chain variable region, a heavy chain constant region, and a first binding domain that can specifically bind PD-L1 and antagonize PD-1 signaling pathway;
ii) a second polypeptide chain, comprising an anti-CD40 light chain variable region;
iii) a third polypeptide chain, comprising, from N terminus to C terminus, an anti-CD40 heavy chain variable region, a heavy chain constant region, and optionally a second binding domain that can specifically bind PD-L1 and antagonize PD-1 signaling pathway; and
iv) a fourth polypeptide chain, comprising an anti-CD40 light chain variable region, wherein the anti-CD40 heavy chain variable region in the first polypeptide chain and the anti-CD40 light chain variable region in the second polypeptide chain associate to form a CD40 binding domain that can specifically bind CD40 and agonize CD40 signaling pathway, the anti-CD40 heavy chain variable region in the third polypeptide chain and the anti-CD40 light chain variable region in the fourth polypeptide chain associate to form a CD40 binding domain that can specifically bind CD40 and agonize CD40 signaling pathway, the heavy chain constant region in the first polypeptide chain and the heavy chain constant region in the third polypeptide chain are associated together.

The CD40 binding domain formed by the first polypeptide chain and the second polypeptide chain, may be the same with or different from, the CD40 binding domain formed by the third polypeptide chain and the fourth polypeptide chain. Alternatively, the two CD40 binding domains may bind the same or different CD40 epitopes.

The first binding domain against PD-L1 may be the same with or different from the second binding domain against PD-L1. Alternatively, the first binding domain and the second binding domain may bind to the same or different PD-L1 epitopes. For example, the first binding domain may be from antibody 3C2, and the second binding domain may be from antibody 56E5; or the first binding domain may be from antibody 56E5, and the second binding domain may be from antibody 3C2. Further, the first binding domain may be from antibody 3C2, and the second binding domain may be an anti-PDL1 nanobody binding to a different epitope; or the second binding domain may be from antibody 3C2, and the first binding domain may be an anti-PDL1 nanobody binding to a different epitope. The antibody 3C2 may be a humanized antibody 3C2VH6VL5, and the antibody 56E5 may be a humanized antibody 56E5VH5VL4.

The first binding domain against PD-L1 may be a single-chain variable fragment (scFv) or a nanobody, and the second binding domain may be a single-chain variable fragment (scFv) or a nanobody. For example, the first binding domain may be a scFv of 3C2VH6VL5 the second binding domain may be an anti-PD-L1 nanobody; or the first binding domain may be an anti-PD-L1 nanobody, the second binding domain may be a scFv of 3C2VH6VL5. Further, the first binding domain may be a scFv of 3C2VH6VL5, and the second binding domain may be a scFv of 56E5VH5VL4 specific to a different epitope; or the first binding domain may be a scFv of 56E5VH5VL4, and the second binding domain may be a scFv of 3C2VH6VL5.

In certain embodiments, the bi- or multi-specific antibody of the disclosure may comprise a first binding domain and a second binding domain specific to PD-L1. The first binding domain may be the same with or different from the second binding domain, and may bind a PD-L1 epitope that is the same with or different from the epitope the second binding domain binds. The first and second binding domains may be a scFv and a nanobody, respectively, both scFvs, or both nanobodies.

The heavy chain constant region in the first and third polypeptide chains may be with weak or no FcR binding affinity, preferably with no FcR binding affinity, such as human IgG1 constant region (N297A), human IgG1 constant region (L234A+L235A), human IgG1 constant region (L234A+L235A+P329G/A), human IgG1 constant region (L234A+L235A+N297A), human IgG1 constant region (L234A+L235A+N297A+P329G/A), human IgG2 constant region (V234A+V237A), and human IgG1 constant region (L234A+V235E), or human IgG4 constant region. In certain embodiments, the heavy chain constant region may comprise the amino acid sequence of SEQ ID NO: 46.

The heavy chain constant region in the first polypeptide chain may be a knob variant, such as human IgG1 or IgG4 constant region or a fragment thereof with T366W mutation. The heavy chain constant region in the first polypeptide chain may be a knob variant with weak or no FcR binding affinity, such as the one comprising the amino acid sequence of SEQ ID NO: 47. The heavy chain constant region in the third polypeptide chain may be a hole variant, such as human IgG1 constant region or a fragment thereof with T366S/L368A/Y407V mutations. The heavy chain constant region in the third polypeptide chain may be a hole variant with weak or no FcR binding affinity, such as the one comprising the amino acid sequence of SEQ ID NO: 48.

Alternatively, the heavy chain constant region in the first polypeptide chain may be a hole variant, such as human IgG1 or IgG4 constant region or a fragment thereof with T366S/L368A/Y407V mutations. The heavy chain constant region in the first polypeptide chain may be a hole variant with weak or no FcR binding affinity, such as the one comprising the amino acid sequence of SEQ ID NO: 48. The heavy chain constant region in the third polypeptide chain may be a knob variant, such as human IgG1 or IgG4 constant region or a fragment thereof with T366W mutation. The heavy chain constant region in the third polypeptide chain may be a knob variant with weak or no FcR binding affinity, such as the one comprising the amino acid sequence of SEQ ID NO: 47.

The second polypeptide chain and/or the fourth polypeptide chain may further comprise a light chain constant region at the C terminus, such as human K light chain constant region. In certain embodiments, the light chain constant region may comprise the amino acid sequence of SEQ ID NO: 6.

The heavy chain constant region in the first polypeptide chain and/or the third polypeptide chain may be linked, directly or via a linker, to the PD-1.1 binding domain, e.g., the first binding domain and/or the second binding domain. The linker may be a peptide of about 5 to 30 amino acid residues. In certain embodiments, the linker may be a peptide of about 5 to 20 amino acid residues. In certain embodiments, the linker may be a GS linker comprising the amino acid sequence of e.g., SEQ ID NOs: 19, 20, 21 or 22.

When the first binding domain and/or the second binding domain against PD-L1 is/are the scFv(s), the heavy chain variable region and the light chain variable region in the scFv may be linked directly or via a linker. The linker may be a peptide of about 5 to 30 amino acid residues. In certain embodiments, the linker may be a peptide of about 5 to 20 amino acid residues. In certain embodiments, the linker may be a GS linker comprising the amino acid sequence of e.g., SEQ ID NOs: 19, 20, 21 or 22.

When the first binding domain and/or the second binding domain against PD-L1 is/are the scFv(s), the heavy chain variable region or the light chain variable region of the scFv may be linked the heavy chain constant region. In certain embodiments, the first binding domain and/or the second binding domain may comprise, from N terminus to C terminus, the heavy chain variable region, an optional linker, and the light chain variable region. In certain embodiments, the first binding domain and/or the second binding domain may comprise, from the N terminus to C terminus the light chain variable region, an optional linker and the heavy chain variable region.

In certain embodiments, the bi- or multi-specific antibody of the disclosure may comprise:
i) a first polypeptide chain comprising, from N terminus to C terminus, an anti-CD40 heavy chain variable region, a heavy chain constant region, a linker, and a scFv or nanobody that can specifically bind PD-L1 and antagonize PD-1 signaling pathway, wherein the scFv may comprise, from N terminus to C terminus, a heavy chain variable region, a linker and a light chain variable region, or alternatively a light chain variable region, a linker and a heavy chain variable region;
ii) a second polypeptide chain comprising, from N terminus to C terminus, an anti-CD40 light chain variable region, and optionally a light chain constant region;
iii) a third polypeptide chain comprising, from N terminus to C terminus, an anti-CD40 heavy chain variable region and a heavy chain constant region; and
iv) a fourth polypeptide chain comprising, from N terminus to C terminus, an anti-CD40 light chain variable region and optionally a light chain constant region.

In certain embodiments, the third polypeptide chain may further comprise at the C terminus a linker and a scFv or nanobody that can specifically bind PD-L1 and antagonize PD-1 signaling pathway, wherein the scFv may comprise, from N terminus to C terminus, a heavy chain variable region, a linker and a light chain variable region, or alternatively a light chain variable region, a linker and a heavy chain variable region. The first binding domain in the first polypeptide chain and the second binding domain in the third polypeptide chain may bind to different PD-L1 epitopes. In certain embodiments, the first polypeptide chain may comprise a scFv that can specifically bind PD-L1 and antagonize PD-L1 signaling pathway, the third polypeptide chain may comprise a scFv that can specifically bind PD-L1 and antagonize PD-L1 signaling pathway. In certain embodiments, the first polypeptide chain may comprise a scFv that can specifically bind PD-L and antagonize PD-L1 signaling pathway, the third polypeptide chain may comprise a nanobody that can specifically bind PD-L1 and antagonize PD-L1 signaling pathway.

The anti-PD-L1 scFv may comprise a VH-CDR1, a VH-CDR2, a VH-CDR3 comprising the amino acid sequences of SEQ ID NOs: 33, 34 and 35, respectively, and a VL-CDR1, a VL-CDR2 and a VL-CDR3 comprising the amino acid sequences of SEQ ID NOs: 36, 37 and 38, respectively. In certain embodiments, the anti-PD-L1 scFv may comprise a heavy chain variable region and a light chain variable region that may comprise amino acid sequences having at least 85%, 86%, 87%, 88%, 89%, 90%, 91%, 92%, 93%, 94%, 95%, 96%, 97%, 98%, 99% or 100% sequence identity to SEQ ID NOs: 3 and 4, respectively.

The anti-PD-L1.1 scFv may comprise a VH-CDR1, a VH-CDR2, a VH-CDR3 comprising the amino acid sequences of SEQ ID NOs: 39, 40 and 41, respectively, and a VL-CDR1, a VL-CDR2 and a VL-CDR3 comprising the amino acid sequences of SEQ ID NOs: 42, 43 and 44, respectively. In certain embodiments, the anti-PD-L1 scFv may comprise a heavy chain variable region and a light chain variable region that may comprise amino acid sequences having at least 85%, 86%, 87%, 88%, 89%, 90%, 91%, 92%, 93%, 94%, 95%, 9%, 97%, 98%, 99% or 100% sequence identity to SEQ ID NOs: 10 and 11, respectively.

The anti-PD-L1 nanobody may comprise a CDR1, a CDR2, and a CDR3 comprising the amino acid sequences of SEQ ID NOs: 49, 50 and 51, respectively. In certain embodiments, the anti-PD-L1 nanobody may comprise an amino acid sequence having at least 85%, 86%, 87%, 88%, 89%, 90%, 91%, 92%, 93%, 94%, 95%, 96%, 97%, 98%, 99% or 100% sequence identity to SEQ ID NO: 52.

The anti-CD40 heavy chain variable region in the first polypeptide chain may comprise a VH-CDR1 and a VH-CDR2 comprising amino acid sequences of SEQ ID NOs: 28 and 29, respectively, and a VH-CDR3 comprising the amino acid sequence of ‘LDY’, and the anti-CD40 light chain variable region in the second polypeptide chain may comprise a VL-CDR1, a VL-CDR2 and a VL-CDR3 comprising the amino acid sequences of SEQ ID NOs: 30, 31 and 32, respectively.

The anti-CD40 heavy chain variable region in the third polypeptide chain may comprise a VH-CDR1 and a VH-CDR2 comprising amino acid sequences of SEQ ID NOs: 28 and 29, respectively, and a VH-CDR3 comprising the amino acid sequence of ‘LDY’, and the anti-CD4) light chain variable region in the fourth polypeptide chain may comprise a VL-CDR1, a VL-CDR2 and a VL-CDR3 comprising the amino acid sequences of SEQ ID NOs: 30, 31 and 32, respectively.

The anti-CD40 heavy chain variable region and the light chain variable region may comprise amino acid sequences having at least 85%, 86%, 87%, 88%, 89%, 90%, 91%, 92%, 93%, 94%, 95%, 96%, 97%, 98%, 99% or 100% sequence identity to SEQ ID NOs: 1 and 2, respectively.

In certain embodiments, the first, second, third and fourth polypeptide chains may comprise amino acid sequences having at least 85%, 86%, 87%, 88%, 89%, 90%, 91%, 92%, 93%, 94%, 95%, 96%, 97%, 98%, 99% or 100% sequence identity to
i) SEQ ID NOs: 17, 53, 16 and 53, respectively;
ii) SEQ ID NOs: 9, 53, 9 and 53, respectively;
iii) SEQ ID NOs: 12, 53, 13 and 53, respectively;
iv) SEQ ID NOs: 18, 53, 18 and 53, respectively;
v) SEQ ID NOs: 14, 53, 13 and 53, respectively;
vi) SEQ ID NOs: 15, 53, 16 and 53, respectively; or
vii) SEQ ID NOs: 12, 53, 16 and 53, respectively.

The bi- or multi-specific antibody of the disclosure may specifically bind CD40 and PD-L1, and may only activate CD40 signaling pathway to induce dendritic cell maturation and trigger T cell activation when binding to the PD-L1 molecules, providing excellent anti-tumor effect with reduced hepatotoxicity. In addition, the bi- or multi-specific antibody of the disclosure may synergize with an anti-PD-L1 antibody such as Tecentriq or an anti-PD-1 antibody such as Keytruda in inducing T cell activation.

A nucleic acid molecule encoding the bi- or multi-specific antibody of the disclosure, is also encompassed by the disclosure, as well as an expression vector that may comprise the nucleic acid molecule and a host cell that may comprise the expression vector or have the nucleic acid molecule integrated in its genome. A method for preparing the bi- or multi-specific antibody of the disclosure using the host cell is also provided, that may comprise steps of (i) expressing the bi- or multi-specific antibody in the host cell and (ii) isolating the bi- or multi-specific antibody from the host cell or its cell culture.

A composition, e.g., a pharmaceutical composition, that may comprise the bi- or multi-specific antibody, the nucleic acid molecule, the expression vector, or the host cell and a pharmaceutically acceptable carrier, is also provided. The pharmaceutical composition of the disclosure may further comprise a monospecific anti-PD-L1 antibody such as Tecentriq, and/or a monospecific anti-PD-1 antibody such as Keytruda.

In a second aspect, the disclosure provides a method for treating or alleviating a tumor or an infectious disease in a subject in need thereof, comprising administering to the subject a therapeutically effective amount of the pharmaceutical composition of the disclosure. In certain embodiments, the method may comprise administering the pharmaceutical composition of the disclosure, as well as an anti-PD-1 antibody and/or an anti-PD-L1 antibody.

The tumor may be a solid tumor or a hematological tumor, including, but not limited to, melanoma, lung cancer (e.g., non-small cell lung cancer), renal cell carcinoma, Hodgkin lymphoma, bladder cancer, head and neck cancer, neumendocrine cancer, mantle cell lymphoma, B cell lymphoma (e.g., diffuse large B-cell lymphoma), follicular lymphoma, multiple myeloma, rectal adenocarcinoma, pancreatic cancer, colorectal cancer, gastric cancer, prostate cancer, kidney cancer, ovarian cancer, cervical cancer, breast cancer, and nasopharyngeal cancer.

The infectious disease may be a chronic viral, bacterial, fungal or mycoplasma infection, such as a chronic infection of hepatitis B virus, hepatitis C virus, human immunodeficiency virus (HIV), or simian immunodeficiency virus (SIV).

The disclosure also provides the use of the bi- or multi-specific antibody, the nucleic acid molecule, the expression vector, the host cell, or the pharmaceutical composition of the disclosure in preparation of a medicament for treating or alleviating a tumor or an infectious disease.

The disclosure further provides the use of the bi- or multi-specific antibody, the nucleic acid molecule, the expression vector, the host cell, or the pharmaceutical composition of the disclosure in inducing dendritic cell maturation, and/or T cell activation.

For example, the present disclosure provides a method for promoting dendritic cell maturation, comprising contacting a dendritic cell with the pharmaceutical composition of the disclosure. In certain embodiments, the method may be used to promote dendritic cell maturation in a subject in need thereof, comprising administering to the subject a therapeutically effective amount of the pharmaceutical composition of the disclosure.

The present disclosure also provides a method for inducing T cell activation, comprising contacting a T cell with the pharmaceutical composition of the disclosure. In certain embodiments, the method may be used to activate the T cell in a subject in need thereof, comprising administering to the subject a therapeutically effective amount of the pharmaceutical composition of the disclosure.

All documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference.

### BRIEF DESCRIPTION OF THE DRAWINGS

The following detailed description, given by way of example, but not intended to limit the invention solely to the specific embodiments as described, may best be understood in conjunction with the accompanying drawings.

FIG. 1 is the schematic diagram of structures of three bi- or multi-specific antibodies of the disclosure targeting CD40 and PD-L1, including the multi-specific antibody comprising an anti-CD40 half antibody and an anti-PD-L1 half antibody (A), the multi-specific antibody comprising an anti-PD-L1 full-length antibody and anti-CD40 scFvs (B), and a multi-specific antibody comprising an anti-CD40 full-length antibody and anti-PD-L1 scFvs (C).

FIG. 2 shows the binding activity of the bi- or multi-specific antibodies to HEK293A/human CD40 cells (A) and HEK293A/monkey CD40 cells (B).

FIG. 3 shows the binding activity of the bi- or multi-specific antibodies to HEK293A/human PD-L1 cells (A) and HEK293A/monkey PD-L1 cells (B).

FIG. 4 shows the PD-L1 staining of dendritic cells (A), and the bi- or multi-specific antibody-mediated expression of maturation markers, including CD86 (B), CD80 (C) and CD83 (D), on dendritic cells.

FIG. 5 shows the ability of the monospecific anti-PD-L1 antibody and the bi- or multi-specific antibody of the disclosure to block PD-L1-PD-1 binding.

FIG. 6 shows the monospecific anti-PD-L1 antibody, monospecific anti-CD40 antibody, the combination of the two monospecific antibodies, and the bi- or multi-specific antibody of the disclosure increased APC-mediated IFN-γ secretion by T cells in a concentration dependent manner, wherein dendritic cell maturation was induced by two cytokines and the antibody (A), or by six cytokines (B).

FIG. 7 is the schematic diagram of structures of exemplary bi- or multi-specific antibodies of the disclosure, including a symmetric antibody (A) and an asymmetric antibody (B) targeting a CD40 epitope and a PD-L1 epitope, a symmetric antibody (C) and an asymmetric antibody (F) targeting a CD40 epitope and another/other PD-L1 epitope(s), a long linker-containing antibody (D) and a short linker-containing antibody (E) targeting a CD40 epitope and two PD-L1 epitopes, and an antibody binding to a CD40 epitope and two PD-L1 epitopes with an an-PD-L1 nanobody (G).

FIG. 8 shows the binding activity of the bi- or multi-specific antibodies to HEK293A/human CD40 cells (A-C) and HEK293A/human PD-L1 cells (D-F).

FIG. 9 shows the activity of the bi- or multi-specific antibodies to activate CD40 signaling pathway (A-C) and to block PD-L1-PD-1 binding (D-F).

FIG. 10 shows the ability of the bi- or multi-specific antibodies and a monospecific anti-CD40 antibody to induce dendritic cell maturation, as measured by the expression levels of CD86 (A-C) and CD83 (D-E), the maturation markers.

FIG. 11 shows the bi- or multi-specific antibodies and a monospecific anti-CD40 antibody increased APC-mediated IFN-γ (A), 11-6 (B) and IL-2 (C) secretion by T cells in a concentration dependent manner.

FIG. 12 shows the bi- or multi-specific antibodies and a monospecific anti-CD40 antibody respectively synergized with Tecentriq, a monospecific anti-PD-L1 antibody, in increasing APC-mediated iFN-γ (A), IL-6 (B) and IL-2 (C) secretion by T cells.

FIG. 13 shows the bi- or multi-specific antibodies and a monospecific anti-CD40 antibody respectively synergized with Tecentriq at different concentrations in increasing APC-mediated IFN-γ (A) and IL-2 (B) secretion by T cells; and the bi- or multi-specific antibodies and a monospecific anti-CD40 antibody respectively synergized with Keytruda, a monospecific anti-PD-1 antibody, at different concentrations in increasing APC-mediated IFN-γ (C) and IL-2 (D) secretion by T cells.

FIG. 14 shows the capability of the bi- or multi-specific antibodies MBS307-6 (A), MBS307-9 (B) and MBS307-10 (C) to bind human CD40 and human PD-L1 simultaneously when they were exposed to CD40 first followed by PD-L1, and the capability of the bi- or multi-specific antibodies MBS307-6 (D), MBS307-9 (E) and MBS307-10 (F) to bind human PD-L1 and human CD40 simultaneously when they were exposed to PD-L1 first followed by CD40, as measured by SPR.

FIG. 15 shows the SPR binding curves in a competitive binding assay of the monospecific anti-PD-L1 antibody 56E5VH5VL4/3C2VH6VL5 and a monospecific anti-PD-L1 nanobody, with 56E5VH5VL4 was injected followed by the anti-PD-L1 nanobody (A), or 3C2VH6VL5 was injected followed by the anti-PD-L1 nanobody (B).

FIG. 16 shows the SPR binding curves in a competitive binding assay of the monospecific anti-PD-L1, antibodies and the bi- or multi-specific antibodies, with 3C2VH6VL5 was injected followed by 3C2VH6VL5, an anti-PD-L1 nanobody or MBS307 11 (A), an anti-PD-L1 nanobody was injected followed by 3C2VH6VL5, the anti-PD-L1 nanobody or MBS307-11 (B), or MBS307-11 was injected followed by 3C2VH6VL5, an anti-PD-L1 nanobody or MBS307-11 (C).

FIG. 17 shows the ALT and AST levels in mice 48 hours post administration of MBS307-11, 7B4VH2VL2-mFc, Tecentriq, 7B4VH2VL2-mFc+Tecentriq, or the vehicle control.

FIG. 18 shows the body weight curves of mice treated with MBS307-11, 7B4VH2VL2-mFc. Tecentriq, 7B4VH2VL2-mFc+Tecentriq, or the vehicle control.

FIG. 19 shows the tumor size curves of mice treated with MBS307-11, 7B4VH2VL2-mFc, Tecentriq, 7B4VH2VL2-mFc+Tecentriq, or the vehicle control.

### DETAILED DESCRIPTION OF THE INVENTION

To ensure that the present disclosure may be more readily understood, certain terms are first defined. Additional definitions are set forth throughout the detailed description.

The term “CD40” refers to tumor necrosis factor receptor superfamily member 5.

The term may comprise variants, isoforms, homologs, orthologs and paralogs. The term “human CD40” refers to a CD40 protein having an amino acid sequence from a human, such as the amino acid sequence having Genbank accession no: NP_001241.1 or set forth in SEQ ID NO:25. The terms “monkey CD40” and “mouse CD40” refer to monkey and mouse CD40 sequences, respectively, e.g., the amino acid sequence having Genbank accession no: NP_001252791.1 or set forth in SEQ ID NO: 26, and the amino acid sequence having Genbank accession no: NP_035741.2 or set forth in SEQ ID NO: 27, respectively.

The term “PD-L1” refers to programmed death-ligand 1. The term may comprise variants, isoforms, homologs, orthologs and paralogs. The term “human PD-L1” refers to a PD-L1 protein having an amino acid sequence from a human, such as the amino acid sequence having Genbank accession no: AAI13735.1 (Strausberg R. L. et al., (2002) Proc. Natl. Acad. Sci. U.S.A. 99(26): 16899-16903) or set forth in SEQ ID NO: 23. The term “monkey PD-L1” refers to a monkey PD-L1 sequence, e.g., the amino acid sequence having NCBI accession no: XP_005581836.1 or set forth in SEQ ID NO: 24.

The term “antibody” as referred to herein includes IgG, IgA, IgD, IgE and IgM whole antibodies and any antigen binding fragment (i.e., “antigen-binding portion”) or single chains thereof. The whole antibodies or full-length antibodies are glycoproteins comprising at least two heavy (H) chains and two light (L) chains inter-connected by disulfide bonds. Each heavy chain is comprised of a heavy chain variable region (abbreviated herein as VW) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions or the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The heavy chain constant region of the disclosure has been designed with weak or no binding affinity to immune cells or the complement system proteins.

It has been shown that the antigen-binding function of an antibody can be performed by fragments of a full-length antibody. Examples of binding fragments encompassed within the term “antigen binding fragment” or “antigen-binding portion” of an antibody include (i) a Fab fragment, a monovalent fragment consisting of the V_{L}, V_{H}, C_{L} and C_{H1} domains; (ii) a F(ab′)2 fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward el al., (1989) Nature 341:544-546), which consists of a V_{H} domain; (vi) an isolated complementarity determining region (CDR); and (vii) a nanobody, a heavy chain variable region containing a single variable domain and two constant domains. The term “IgG like antibody” refers to an antibody with the basic structure of an IgG antibody and additional functional moieties such as an antigen binding domain.

The “knob variant” of a heavy chain constant region, or a heavy chain constant region with “knob mutation(s)” refers to a heavy chain constant region used in the knobs-into-holes technology whose CH3 domains are engineered to create a “knob”. Similarly, the “hole variant” of a heavy chain constant region, or a heavy chain constant region with “hole mutation(s)” refers to a heavy chain constant region used in the knobs-into-holes technology whose CH3 domains are engineered to create a “hole”.

The term “agonistic anti-CD40 antibody” refers to an anti-CD40 antibody that can specifically hind CD40 and activate or induce CD40 signaling pathway to promote immune cell activation and proliferation, and production of cytokines and chemokines. In contrast, an “antagonistic and-CD40 antibody” may block or inhibit the CD40 signaling pathway that may be triggered by CD40L engagement. The CD40 binding domain in the bi- or multi-specific antibodies of the disclosure may be agonistic, i.e., it can bind CD40 and activate CD40 signaling pathway.

The term “antagonistic anti-PD-L1 antibody” or “PD-L1 blocking antibody” refers to an anti-PD-L1 antibody that can block or inhibit the PD-1 signaling triggered by PD-L1 engagement with PD-1. The antagonistic anti-PD-L1 antibody may promote T cell activation and cytokine release, and enhance immune responses, and thus can be used to treat cancers and chronic infections. The PD-L1 binding domain in the bi- or multi-specific antibodies of the disclosure may be antagonistic, i.e., it can bind PD-L1 and block PD-L1-PD-1 binding or interaction, which does not trigger PD-1 signaling pathway. The term “antagonize PD-1 signaling pathway” or “antagonizing PD-1 signaling pathway” means the PD-1 binding domain blocks PD-L1-PD-1 binding or interaction and does not trigger PD-1 signaling.

The term “FcR” or “Fe receptor” refers to a protein expressed on the surface of certain immune cells such as B lymphocytes, natural killer cells, and macrophages, which recognizes the Fc fragment of antibodies that are attached to cells or pathogens, and stimulates phagocytic or cytotoxic cells to destroy pathogens or target cells by e.g., antibody-mediated phagocytosis or antibody-dependent cell-mediated cytotoxicity. The FcR includes, FcαR, FcεR and FcγR, and the FcγR belongs to the immunoglobulin superfamily and is the most important Fc receptor for inducing phagocytosis of microbes, including FcγRI (CD64), FcγRIIA (CD32A), FcγRIIB (CD32B), and FcγRIIIA (CD16A).

A “bi-specific” or “multi-specific” antibody specifically binds two or more (e.g., three) target molecules, or two or more (e.g., three) different epitopes in a same target molecule. The bi- or multi-specific antibody of the disclosure specifically binds CD40 and PD-L1. In contrast, a “monospecific” molecule specifically binds a certain target molecule, especially a certain epitope in the target molecule.

The term “half antibody” or “half-antibody” refers to one half of a full-length antibody which comprises e.g., a heavy chain and a light chain.

The percent “sequence identity” as used herein in the context of two or more nucleic acids or polypeptides, refers to two or more sequences or subsequences that are the same or have a specified percentage of nucleotides or amino acid residues that are the same, when compared and aligned (introducing gaps, if necessary) for maximum correspondence, considering or not considering conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST. ALIGN. Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection.

As used herein, an antibody that “specifically binds CD40” or “specifically binds PD-L1” is intended to refer to an antibody that binds to CD40 or PD-L1 but does not substantially bind to non-CD40/PD-L1 proteins. Preferably, the antibody binds CD40 or PD-L1 protein with “high affinity”, namely with a K_{D} of 5.0×10⁻⁸ M or less.

The term “does not substantially bind” to a protein or cells, as used herein, means does not bind or does not bind with a high affinity to the protein or cells, i.e., binds to the protein or cells with a K_{D} of 1.0×10⁻⁶ M or more.

The term “epitope” refers to a region on the surface of an antigen that involves in specific binding with a receptor or an antibody specific to the antigen. The “PD-L1 epitope” herein refers to a region on the surface of a PD-L1 protein that is recognized and bound by an anti-PD-L1 antibody. An antigen may have one or multiple epitopes, wherein two epitopes may be totally separated and different in structure and sequence, and may also partially overlap in structure and sequence (referred to as overlapping epitopes). Therefore, when two antibodies recognize and bind to an antigen at “different epitopes”, they may bind to different sites of a same antigen simultaneously, or alternatively only one of them may bind to the antigen as the binding of this antibody with the antigen hinders the binding of the other antibody to the antigen. Antibodies binding to the same epitope or overlapping epitopes may compete over antigen binding.

The term “EC₅₀”, also known as half maximal effective concentration, refers to the concentration of an antibody which induces a response halfway between the baseline and maximum after a specified exposure time.

The term “IC₅₀”, also known as half maximal inhibitory concentration, refers to the concentration of an antibody which inhibits a specific biological or biochemical function by 50% relative to the absence of the antibody.

The term “cross-link” or “cross-linking” herein refers to aggregation of the antibodies through binding of the antibodies' Fe regions to FcRs on immune cells, or through binding of the antibodies via the PD-L1 binding domains to PD-L1s on e.g., tumor cells. In in vitro tests, antibody cross-linking may occur when antibodies bind their Fc regions to the anti-Fc secondary antibodies coupled to e.g., E LISA plates. The anti-CD40 antibody or the antigen-binding fragment thereof of the disclosure may only promote dendritic cell maturation and T cell activation when antibody cross-linking occurs. In contrast, “free” antibodies or antigen-binding fragments thereof refer to those that do not interact or have not interacted to form dimers, trimers or polymers. The free anti-CD40 antibodies or the antigen binding fragments thereof of the disclosure do not active T cells.

The term “subject” includes any human or nonhuman animal. The term “non-human animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “effective amount” refers to an amount of the antibody of the disclosure sufficient to achieve the desired effects. The term “therapeutically effective amount” means an amount of the antibody of the present disclosure sufficient to prevent or ameliorate the symptoms associated with a disease or condition (such as a cancers) and/or lessen the severity of the disease or condition. A therapeutically effective amount is understood to be in context to the condition being treated, where the actual effective amount is readily discerned by those of skill in the art.

### Advantageous Characteristics of Multi-Specific Antibodies

The bi- or multi-specific antibodies of the disclosure may bind CD40 and PD-L1 simultaneously, and can block PD-1-PD-L1 interaction. With reduced or eliminated FcR binding affinity, non-specific CD40 signaling activation by the bi- or multi-specific antibodies can be decreased, such that cross linking of these antibodies may only occur when they bind PD-L1 molecules, which may activate CD40 signaling pathway, promoting dendritic cell maturation and T cell activation. The bi- or multi-specific antibodies may enhance e.g., the anti-tumor efficacy while reducing toxic side effects.

The inventors of the application, with antibody construction and structure optimization, found that the bi- or multi-specific antibody which comprises a full-length anti-CD40 antibody and an anti-PD-L1 scFv or an anti-PD-L1 nanobody linked to the C terminus of the heavy chain of the anti-CD40 antibody, maintains the PD-L1 and CD40 binding capability, and thus can promote dendritic cell maturation and T cell activation. When the multi-specific antibody comprises an anti-CD40 half antibody and an anti-PD-L1 half antibody, or comprises a full-length anti-PD-L1 antibody and an anti-CD40 scFv linked to the C terminus of the heavy chain of the anti-PD-L1 antibody, is not able to promote dendritic cell maturation or T cell activation.

Further, with the structure as screened out, the multi-specific antibodies, when comprising two anti-PD-L1 scFvs binding different epitopes or alternatively one anti-PD-L1 scFv and one anti-PD-L1.1 nanobody binding different epitopes linked to the C terminus of the heavy chains of the anti-CD40 antibody, show superior effects on dendritic cell maturation, T cell activation and tumor suppression. In the in vivo assays, the tri-specific antibodies showed better anti-tumor effect than the combination of a monospecific anti-CD40 antibody and Tecentriq, with evidently less hepatotoxicity and body weight decrease.

The heavy chain variable region CDRs and light chain variable region CDRs of the monospecific antibodies or antigen binding fragments thereof used in the bi- or multi-specific antibodies of the disclosure have been defined by the Kabat numbering system. However, as is well known in the art, CDRs can also be determined by other systems such as Chothia, and IMGT, AbM, or Contact numbering system/method, based on heavy chain/light chain variable region sequences.

The multi-specific antibody of the disclosure includes the bi-specific antibody.

### Multi-Specific Antibodies

The multi-specific antibody of the disclosure may comprise:
i) a first polypeptide chain, comprising, from N terminus to C terminus, an anti-CD40 heavy chain variable region, a heavy chain constant region, and a first binding domain that can specifically bind PD-L1 and antagonize PD-1 signaling pathway;
ii) a second polypeptide chain, comprising an anti-CD40 light chain variable region;
iii) a third polypeptide chain, comprising, from N terminus to C terminus, an anti-CD40 heavy chain variable region, a heavy chain constant region, and optionally a second binding domain that can specifically bind PD-L1 and antagonize PD-1 signaling pathway; and
iv) a fourth polypeptide chain, comprising an anti-CD40 light chain variable region, wherein the anti-CD40 heavy chain variable region in the first polypeptide chain and the anti-CD40 light chain variable region in the second polypeptide chain associate to form a CD40 binding domain that can specifically bind CD40 and agonize CD40 signaling pathway, the anti-CD40 heavy chain variable region in the third polypeptide chain and the anti-CD40 light chain variable region in the fourth polypeptide chain associate to form a CD40 binding domain that can specifically bind CD40 and agonize CD40 signaling pathway, the heavy chain constant region in the first polypeptide chain and the heavy chain constant region in the third polypeptide chain are associated together via e.g., the knobs-into-holes approach, the covalent bond(s) and/or the disulfide bond(s).

The CD40 binding domain formed by the first polypeptide chain and the second polypeptide chain, may be same with or different from, the CD40 binding domain formed by the third polypeptide chain and the fourth polypeptide chain. Alternatively, the two CD40 binding domains may bind the same or different CD40 epitopes.

The first binding domain against PD-L1 may be the same with or different from the second binding domain against PD-L1. Alternatively, the first binding domain and the second binding domain may bind to the same or different PD-L1 epitopes.

The first binding domain against PD-L1 may be a single-chain variable fragment (scFv) or a nanobody, and the second binding domain may be a single-chain variable fragment (scFv) or a nanobody.

In certain embodiments, the multi-specific antibody of the disclosure may comprise a first binding domain and a second binding domain specific to PD-L1. The first binding domain may be the same with or different from the second binding domain, and may bind a PD-L1 epitope that is the same with or different from the epitope the second binding domain binds. The first and second binding domains may be a scFv and a nanobody, respectively, both scFvs, or both nanobodies.

In an embodiment, the multi-specific antibody of the disclosure may comprise:
i) a first polypeptide chain comprising, from N terminus to C terminus, an anti-CD40 heavy chain variable region, a heavy chain constant region, a linker, and a scFv that can specifically bind PD-L1 and antagonize PD-1 signaling pathway, wherein the scFv may comprise, from N terminus to C terminus, a heavy chain variable region, a linker and a light chain variable region, or alternatively a light chain variable region, a linker and a heavy chain variable region;
ii) a second polypeptide chain comprising, from N terminus to C terminus, an anti-CD40 light chain variable region, and optionally a light chain constant region;
iii) a third polypeptide chain comprising, from N terminus to C terminus, an anti-CD40 heavy chain variable region and a heavy chain constant region; and
iv) a fourth polypeptide chain comprising, from N terminus to C terminus, an anti-CD40 light chain variable region and optionally a light chain constant region.

In an embodiment, the multi-specific antibody of the disclosure may comprise:
i) a first polypeptide chain comprising, from N terminus to C terminus, an anti-CD40 heavy chain variable region, a heavy chain constant region, a linker, and a scFv that can specifically bind PD-L1 and antagonize PD-1 signaling pathway, wherein the scFv may comprise, from N terminus to C terminus, a heavy chain variable region, a linker and a light chain variable region;
ii) a second polypeptide chain comprising, from N terminus to C terminus, an anti-CD40 light chain variable region, and optionally a light chain constant region;
iii) a third polypeptide chain comprising, from N terminus to C terminus, an anti-CD40 heavy chain variable region, a heavy chain constant region, a linker, and a scFv or a nanobody that can specifically bind PD-L1 and antagonize PD-1 signaling pathway, wherein the scFv may comprise, from N terminus to C terminus, a heavy chain variable region, a linker and a light chain variable region; and
iv) a fourth polypeptide chain comprising, from N terminus to C terminus, an anti-CD40 light chain variable region and optionally a light chain constant region.

### Linkers

The heavy chain constant region in the first and/or third polypeptide chain(s) may be linked via a linker to the PD-L1 binding domain, e.g., the first binding domain and/or the second binding domain specific to PD-L1. When the first and/or second binding domain(s) specific to PD-L1 is/are the scFv(s), the heavy chain variable region and the light chain variable region in the scFv(s) may be linked via a linker.

The linker may be made up of amino acids linked together by peptide bonds, preferably from 5 to 30 amino acids linked by peptide bonds, wherein the amino acids are selected from the 20 naturally occurring amino acids. One or more of these amino acids may be glycosylated, as is understood by those of skill in the art. In one embodiment, the 5 to 30 amino acids may be selected from glycine, alanine, proline, asparagine, glutamine, serine and lysine. In one embodiment, a linker is made up of a majority of amino acids that are sterically unhindered, such as glycine and alanine. Exemplary linkers are polyglycines, particularly poly(Gly-Ala), and polyalanines. The exemplary linker as used herein may comprise the amino acid sequence of SEQ ID NOs: 19, 20, 21 or 22.

The linker may also be a non-peptide linker. For example, alkyl linkers such as —NH—, —(CH₂)s-C(O)—, wherein s=2-20 can be used. These alkyl linkers may further be substituted by any non-sterically hindering group such as lower alkyl (e.g., C₁₋₆) lower acyl, halogen (e.g., Cl, Br), CN, NIH, phenyl, etc.

### Conservative Modifications

The multi-specific antibody of the disclosure may comprise a heavy chain variable region and/or a light chain variable region or the CDR1, CDR2 and CDR3 sequences with one or more conservative modifications. It is understood in the art that certain conservative sequence modification can be made which does not remove antigen binding capability.

As used herein, the term “conservative sequence modification” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), non-polar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the disclosure can be replaced with other amino acid residues from the same side chain family and the altered antibody can be tested for retained function (i.e., the functions set forth above) using the functional assays described herein.

### Engineered Antibodies

The multi-specific antibody of the disclosure can be prepared using an antibody having one or more of the V_{H}/V_{L}, sequences of the present disclosure, as starting material to engineer a modified antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

Therefore, the heavy and/or light chain variable region(s) in the multi-specific antibodies of the disclosure may contain the VH-CDR1. VH-CDR2, and VH-CDR3, and/or the VL-CDR1, VL-CDR2 and VL-CDR3, but different framework regions.

The inventors of the application found that, when an antigen binding domain is in the scFv format, the conformational stability may be lower than that of the full-length antibody or the Fab format. Therefore, in one embodiment of the disclosure, to solve the anti-PD-L1 scFv's conformational stability problem, the inventors of the application, with computer modeling, made modifications on the frameworks of the heavy/light chain variable regions, e.g., the FR3 and/or FR4 regions of light chain variable region, so as to enhance scFv's conformational stability, reducing the formation of aggregates, i.e., increasing monomer levels. The Fab and scFv with the modified framework(s) showed comparable target binding affinity, PD-L1-PD-1 blocking capability and anti-tumor efficacy.

The inventors of the application further found that, the multi-specific antibodies may be further improved in structure stability and production levels in industrial applications by replacing the hydrophobic amino acid residues with hydrophilic or less hydrophobic ones, or versa vice, in the framework region(s) of the anti-PD-L1 heavy/light chain variable region(s), based on computer modeling.

Engineered antibodies of the disclosure include those in which modifications have been made to framework residues within V_{H} and/or V_{L}, to change the antibodies' characteristic. Typically, the framework regions are modified to reduce the potential immunogenicity. One approach is to “back mutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043.

In addition, or as an alternative to modifications made within the framework or CDR regions, the antibodies of the disclosure can be engineered to include modifications within the Fc region, typically to alter one or more functional properties, such as serum half-life, complement fixation, Fc receptor binding, and/or antigen-dependent cellular cytotoxicity. Furthermore, the antibodies of the disclosure can be chemically modified (e.g., one or more chemical moieties can be attached to the antibody) or be modified to alter its glycosylation, again to alter one or more functional properties.

In one embodiment, the hinge region of Cm is modified such that the number of cysteine residues in the hinge region is altered, e.g., increased or decreased. This approach is described further in U.S. Pat. No. 5,677,425. The number of cysteine residues in the hinge region of Cm is altered to, for example, facilitate assembly of the light and heavy chains or to increase or decrease the antibody stability.

In another embodiment, the Fe hinge region of an antibody is mutated to decrease the biological half-life. More specifically, one or more amino acid mutations are introduced into the C_{H2}-C_{H3} domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In still another embodiment, the glycosylation of the antibodies is modified. For example, a de-glycosylated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation. For example, one or more amino acid substitutions can be made that result in elimination of one or more variable region framework glycosylation sites to thereby eliminate glycosylation at that site. Such aglycosylation may increase the affinity of the antibody for the antigen. See, e.g., U.S. Pat. Nos. 5,714,350 and 6,350,861. Additionally, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of the antibody. Such carbohydrate modifications can be accomplished by, for example, expressing the antibody in a host cell with altered glycosylation machinery. Cells with altered glycosylation machinery have been described in the art and can be used as host cells in which to express the antibody of the disclosure to thereby produce an antibody with altered glycosylation. For example, the cell lines Ms704, Ms75, and Ms709 lack the fucosyltransferase gene, FUT8 (α (1, 6)-fucosyltransferase), such that antibody expressed in the Ms704, Ms705, and Ms709 cell lines lacks fucose on their carbohydrates.

Another modification of the antibodies herein is pegylation. An antibody can be pegylated to, for example, increase the biological (e.g., serum) half-life. To pegylate an antibody, the antibody or the fragment thereof typically is reacted with polyethylene glycol (PEG), such as a reactive ester or aldehyde derivative of PEG, under conditions in which one or more PEG groups become attached to the molecule.

### Nucleic Acid Molecules Encoding Antibodies

In another aspect, the disclosure provides a nucleic acid molecule that encodes the anti-CD40 heavy chain variable region-heavy chain constant region-linker-anti-PD-L1 heavy chain variable region-linker-anti-PD-L1 light chain variable region chain, the anti-CD40 heavy chain variable region-heavy chain constant region-linker-anti-PD-L1 light chain variable region-linker-anti-PD-L1 heavy chain variable region chain, the anti-CD40 heavy chain variable region-heavy chain constant region-linker-anti-PD-L1 nanobody chain, the anti-CD40 heavy chain variable region-heavy chain constant region, anti-CD40 light chain variable region, or anti-CD40 light chain variable region-light chain constant region chain used in the multi-specific antibodies of the disclosure.

The nucleic acid molecule can be present in whole cells, in a cell lysate, or in a partially purified or substantially pure form. A nucleic acid is “isolated” or “rendered substantially pure” when purified away from other cellular components or other contaminants. e.g., other cellular nucleic acids or proteins, by standard techniques. A nucleic acid of the disclosure can be, e.g., DNA or RNA and may or may not contain intronic sequences.

The nucleic acid molecule of the disclosure can be obtained using standard molecular biology techniques. Preferred nucleic acids molecules of the disclosure include those encoding the V_{H} and/or V_{L} sequences of the anti-PD-L1 or anti-CD40 antibody or the CDRs. Once DNA fragments encoding V_{H} and/or V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

To create a scFv gene, the V_{H}- and V_{L}-encoding DNA fragments are operatively linked to another fragment encoding a flexible linker, such as a GS linker, such that the V_{H} and V_{L} sequences can be expressed as a contiguous single-chain protein, with the V_{L} and V_{H} regions joined by the flexible linker.

For the multi-specific antibodies of the disclosure, nucleic acid sequences encoding the anti-CD40 antibody's CDRs, V_{H} and V_{L}, the anti-PD-L1 antibody's V_{H} and V_{L}, and linkers are firstly synthesized, and then combined according to the required structures. For example, the DNA sequences coding for the anti-CD40 heavy chain variable region, the heavy chain constant region, the anti-PD-L1 light chain variable region, the linker, and the anti-PD-L1 heavy chain variable region can be “operatively” linked.

### Generation of Antibodies

The multi-specific antibody of the disclosure may be produced by i) inserting the nucleotide sequences encoding the polypeptide chains of the multi-specific antibody into one or more expression vectors which are operatively linked to regulatory sequences that control transcription or translation; (ii) transducing or transfecting host cells with the expression vectors; and (iii) expressing the polypeptide chains to form the multi-specific antibody of the disclosure.

The term “regulatory sequence” is intended to include promoters, enhancers and other expression control elements (e.g., polyadenylation signals) that control the transcription or translation of the antibody genes.

The expression vector can encode a signal peptide that facilitates secretion of the polypeptide chain(s) from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the polypeptide chain genes and regulatory sequences, the expression vectors of the disclosure can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

The expression vector(s) can be transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the multi-specific antibody of the disclosure in either prokaryotic or eukaryotic host cells, expression of the multi-specific antibody in eukaryotic cells, and most preferably mammalian host cells, is the most preferred because such eukaryotic cells, and in particular mammalian cells, are more likely than prokaryotic cells to assemble and secrete a properly folded and immunologically active molecule.

The expression vectors that can be used in the present application include but are not limited to plasmids, viral vectors, yeast artificial chromosomes (YACs), bacterial artificial chromosomes (BACs), transformation-competent artificial chromosomes (TACs), mammalian artificial chromosomes (MACs) and human artificial episomal chromosomes (HAECs).

### Pharmaceutical Compositions

In another aspect, the present disclosure provides a pharmaceutical composition which may comprise the multi-specific antibody or functional fragment thereof, the nucleic acid molecule, the expression vector, or the host cell, of the disclosure, formulated together with a pharmaceutically acceptable carrier. The pharmaceutical composition may optionally contain one or more additional pharmaceutically active ingredients, such as an anti-tumor antibody, or alternatively a non-antibody anti-tumor agent. The pharmaceutical composition of the disclosure may be used in combination with an additional anti-tumor agent.

The pharmaceutical composition may comprise any number of excipients. Excipients that can be used include carriers, surfactants, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003).

Preferably, the pharmaceutical composition is suitable for intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion. Alternatively, an antibody of the disclosure can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a micro-emulsion, liposome, or other ordered structure suitable to high drug concentration.

The administration of the pharmaceutical composition of the disclosure may be determined by physicians, e.g., doctors, depending on a subject's e.g., sex, age, medical history and the like.

A “therapeutically effective dosage” of the pharmaceutical composition of the disclosure, may result in a decrease in severity of disease symptoms, or an increase in frequency and duration of disease symptom-free periods. For example, for the treatment of tumor-bearing subjects, a “therapeutically effective dosage” preferably reduces tumor size by at least about 20%, more preferably by at least about 40%, even more preferably by at least about 60%, and still more preferably by at least about 80%, or even eliminate tumors, relative to untreated subjects. For the treatment of patients with viral infections, especially chronic infections, a “therapeutically effective dosage” preferably reduces viral RNAs by at least about 20%, more preferably by at least about 40%, even more preferably by at least about 60%, and still more preferably by at least about 80%, or even eliminate viral RNAs, relative to untreated subjects.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid.

In certain embodiments, the antibodies of the disclosure can be formulated to ensure proper distribution in vivo. For example, to ensure that the therapeutic antibody of the disclosure cross the blood-brain barrier, they can be formulated in liposomes, which may additionally comprise targeting moieties to enhance selective transport to specific cells or organs.

### Uses and Methods

The pharmaceutical composition of the disclosure has multiple in vitro and in vivo applications. For example, the pharmaceutical composition may be used to treat or alleviate tumors and infectious diseases.

The pharmaceutical composition of the disclosure may be used to treat or alleviate tumors. The tumor may be a solid tumor or a hematological tumor, including, but not limited to, melanoma, lung cancer (e.g., non-small cell lung cancer), renal cell carcinoma. Hodgkin lymphoma, bladder cancer, head and neck cancer, neuroendocrine cancer, mantle cell lymphoma, B cell lymphoma (e.g., diffuse large B-cell lymphoma), follicular lymphoma, multiple myeloma, rectal adenocarcinoma, pancreatic cancer, colorectal cancer, gastric cancer, prostate cancer, kidney cancer, ovarian cancer, cervical cancer, breast cancer, and nasopharyngeal cancer.

The pharmaceutical composition of the disclosure may be used to treat or alleviate infectious diseases. The infectious disease may be a chronic viral, bacterial, fungal or mycoplasma infection, such as a chronic infection of hepatitis B virus, hepatitis C virus, human immunodeficiency virus (HIV), or simian immunodeficiency virus (SIV).

The pharmaceutical composition of the disclosure may be used to promote dendritic cell maturation and/or T cell activation.

The disclosure provides methods of combination therapy in which the pharmaceutical composition of the present disclosure is co-administered with one or more additional antibodies or non-antibody agents, e.g., anti-PD-1 antibodies, and anti-PD-L1 antibodies, that are effective in treatment or alleviation of certain diseases.

Various aspects and embodiments of the disclosure will be discussed in reference to the drawings and the following Examples. Other aspects and embodiments are apparent to those skilled in the art. All references described herein are incorporated herein by reference in their entirety. Although the present application has been described in reference to the exemplary embodiments, many changes and alterations, in view of the disclosure, are apparent to those skilled in the art. Therefore, the exemplary embodiments of the disclosure are only provided for illustration, and should not been construed as limiting. Various alterations may be made to the embodiments without departing from the sprit and scope of the application.

### EXAMPLES

### Example 1. Construction of Cell Lines Stably Expressing PD-L1 or CD40

Cell lines stably expressing human and monkey PD-L1 proteins were constructed using HEK293A cells. Briefly, the sequences encoding human and monkey PD-L1 proteins (amino acid sequences set forth in SEQ ID NOs: 23 and 24, respectively) were synthesized, and then subcloned into pLV-EGFP(2A)-Puro vectors (Beijing Inovogen, CN) between EcoRI and HindIII. Lentiviruses were generated in HEK293T cells (Cobioer, NJ, CN) by cotransfection of the resultant pLV-EGFP(2A)-Puro-PD-L1, psPAX and pMD2.G plasmids, according to the instruction in Lipofectamine 3000 kit (Thermo Fisher Scientific, USA). Three days post cotransfection, the lentiviruses were harvested from the HEK293T cell culture medium (DMEM (Cat #:SH30022.01, Gibco) supplemented with 10% FBS (Cat #:FND500, Excell), and then used to infect HEK293A cells (Cobioer, NJ, CN), so as to generate HEK293A cells stably expressing human or monkey PD-L1, namely HEK293A/human PD-L1 and HEK293A/monkey PD-L1 cells, respectively. These HEK293A cells were cultured in DMEM containing 10% FBS and 0.2 μg/ml puromycin (Cat #:A11138-03, Gibco) for 7 days. The expression of human and monkey PD-L1s was confirmed by FACS using commercially available anti-PD-L1 antibodies (PE anti-human PD-L1 Antibody, Cat #:393607, Biolegend, USA).

Similarly, cell lines stably expressing human, monkey or mouse CD40 protein (amino acid sequences set forth in SEQ ID NOs: 25, 26 and 27, respectively) were constructed using HEK293A cells (see Example 1 of WO2020177321A1). The expression of human and monkey CD40 proteins was tested by FACS using commercially available anti-human CD40 antibodies (PE anti-human CD40 Antibody, Cat #:313006, Biolegend, USA), while the mouse CD40 expression was confirmed by FACS using commercially available anti-mouse CD40 antibodies (PE anti-mouse CD40 Antibody. Cat #:124609, Biolegend, USA)

### Example 2. Construction and Expression of Anti-CD401PD-L1 Bispecific Antibodies

Multiple bispecific antibodies were constructed as full-length antibodies with the structures shown in FIG. 1. The CD40 binding domain used a heavy chain variable region and a light chain variable region comprising the amino acid sequences of SEQ ID NOs: 1 and 2, respectively (i.e., the heavy and light chain variable regions of the anti-CD40 antibody 7B4VH2VL2, see WO2020177321A1), while the PD-L1 binding domain employed a heavy chain variable region and a light chain variable region comprising the amino acid sequences of SEQ ID NOs: 3 and 4, respectively (i.e., the heavy and light chain variable regions of the anti-PD-L1 antibody 56E5VH-5VL4).

Two half antibodies MBS307-1-1 (comprising the polypeptide chain of SEQ ID NO: 5 containing anti-CD40 heavy chain variable region-knob variant of heavy chain constant region-linker 1-TGFBII, the polypeptide chain of SEQ ID NO: 53 containing anti-CD40 light chain variable region-light chain constant region) and MBS307-1-2 (comprising the polypeptide chain of SEQ ID NO: 7 containing anti-PD-L1 heavy chain variable region (56E5VH5VL4)-hole variant of heavy chain constant region-linker 1-TGFBII, anti-PD-L1 light chain variable region (56E5VH5VL4) of SEQ ID NO: 4, light chain constant region of SEQ ID NO: 6) for the asymmetrical anti-CD40/PD-L1 antibody MBS307-1 were constructed, as well as two full-length antibodies MBS307-2 (comprising the polypeptide chain of SEQ ID NO: 8 containing anti-PD-L1 heavy chain variable region (56E5VH5VL4)-heavy chain constant region-linker 2-anti-CD40 heavy chain variable region-linker 1-anti-CD40 light chain variable region, anti-PD-L1 light chain variable region of SEQ ID NO: 4, light chain constant region of SEQ ID NO: 6) and MBS307-3 (comprising the polypeptide chain of SEQ ID NO: 9 containing anti-CD40 heavy chain variable region-heavy chain constant region-linker 2-anti-PD-L1 heavy chain variable region (56E5VH5VL4)-linker 1-anti-PD-L1 light chain variable region (56E5VH5VL4), the polypeptide chain of SEQ ID NO: 53 containing anti-CD40 light chain variable region-light chain constant region) as the symmetrical bispecific antibodies.

DNA sequences encoding the long (heavy) and short (light) chains of the two symmetric antibodies were synthesized, and digested with EcoRI and Nhel. The resultant DNA fragments were inserted into the expression vectors. Similarly, the DNA sequences encoding the long (heavy) and short (light) chains of the two half antibodies were synthesized, digested with restriction enzymes, and the resultant fragments were inserted into the expression vectors. The expression vectors with the correct sequences were obtained and designated as GS-MBS307-1-1, GS-MBS307-1-2, GS-MBS307-2 and GS-MBS307-3.

HEK-293F cells (Cobioer. CN) were transfected with the expression vectors obtained above using PEI. Briefly, the HEK-293F cells were cultured in Free Style™ 293 expression medium (Cat #:12338-018. Gibco), and transfected with the expression vectors using polyethyleneinimine (PEI) at a DNA:PEI ratio of 1:3, 1.5 μg of DNAs per millimeter of cell culture medium. The transfected HEK-293F cells were cultured in an incubator at 37° C. under 5% CO₂ with shaking at 120 RPM. After 10-12 days, the cell culture supernatants were harvested, centrifuged at 3500 rpm for 5 min, and flowed through a 0.22 μm film filter to remove the cell debris. The half antibodies and the full-length antibodies as expressed were purified using pre-equilibrated Protein-A affinity columns (Cat #:17040501, GE, USA) and eluted with the elution buffer (20 mM citric acid, pH 3.0-3.5). The antibodies were kept in PBS buffer (pH 7.0) and the concentrations were determined using a NanoDrop analyzer.

### Example 3. Assembly of Asymmetrical Antibodies

The purified half-antibodies were assembled in vitro. Briefly, the two half antibodies, MBS307-1-1 and MBS307-1-2, were mixed at 1:1 molar ratio. The mixtures were added with Tris base buffer till pH 8.0 followed by reducing glutathione (GSH), and allowed to react overnight at 25° C. with low-speed stirring. Then, the mixtures were added with 2 M acetic acid solution to adjust pH to 5.5. The reducing GSH was removed by ultrafiltration, to terminate the reaction. The antibodies were purified using anions exchange chromatography followed by cation exchange chromatography. Anion exchange columns were balanced with low-salt Tris buffer (pH8.0), and loaded with the antibody samples. The components that had passed through the columns were collected, and rinsed by low-salt Tris buffer (pH8.0) until UV280 trended to the baseline. The collected samples were adjusted to pH5.5 using an acetic acid solution, concentrated to 1 ml using a 30 kDa ultrafilter tube, and filtered using 0.2 μm membrane. Then, cation exchange columns were balanced with a low-concentration acetate buffer (pH5.5), and loaded with the antibody samples. The low-concentration acetate buffer (pH5.5) was used to balance the columns again, and elution was done using 20 CV acetate solutions (concentration at 0-100%, pH5.5).

The bispecific antibody as assembled was designated as MBS307-1. The purified antibodies with a purity higher than 90% as measured by mass spectrum, were further characterized below.

### Example 4. Binding Capability of Bispecific Antibodies to Human CD40. Monkey CD40 and Mouse CD40

The bispecific antibodies as prepared above were tested for their binding capability to cell surface human, monkey and mouse CD40 proteins by FACS, using the HEK293A cells prepared in Example 1 that stably expressed human, monkey or mouse CD40. Briefly, a 96-well plate was seeded with 10⁵ HEK293A cells in 50 μl PBS, and then added with 100 μl serially diluted anti-CD40 antibody 714VH2VL2 and the bispecific antibodies of the disclosure (starting concentration at 40 μg/ml). After incubation at 4° C. for 1 h, the plate was washed by PBST for 3 times. The plate was added with 1:500 diluted APC-goat anti-mouse IgG (Cat #:405308, BioLegen, USA), incubated at 4° for 1 h, washed with PBS for 3 times, and subjected to fluorescence measurement in a FACS machine (BD).

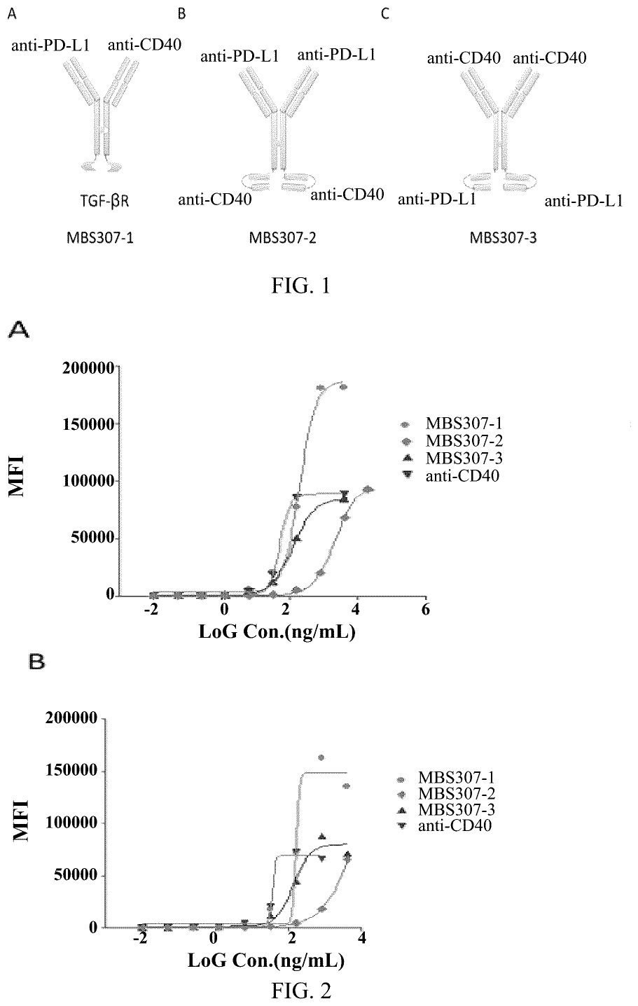

The results were shown in FIG. 2. All bispecific antibodies bound human and monkey CD40s but did not bind mouse CD40. MBS307-2 showed evidently decreased binding capability to human and monkey CD40s than the monospecific anti-CD40 antibody, while MBS307-3 showed close, particularly a bit lower, human and monkey CD40 binding capability compared to the monospecific anti-CD40 antibody.

### Example 5. Binding Capability of Bispecific Antibodies to Human PD-L1 and Monkey PD-L1

Following the protocol of Example 4, the bispecific antibodies were tested for their binding capability to cell surface human, or monkey PD-L1 proteins by FACS, using the HEK293A cells prepared in Example 1 that stably expressed human or monkey PD-1.

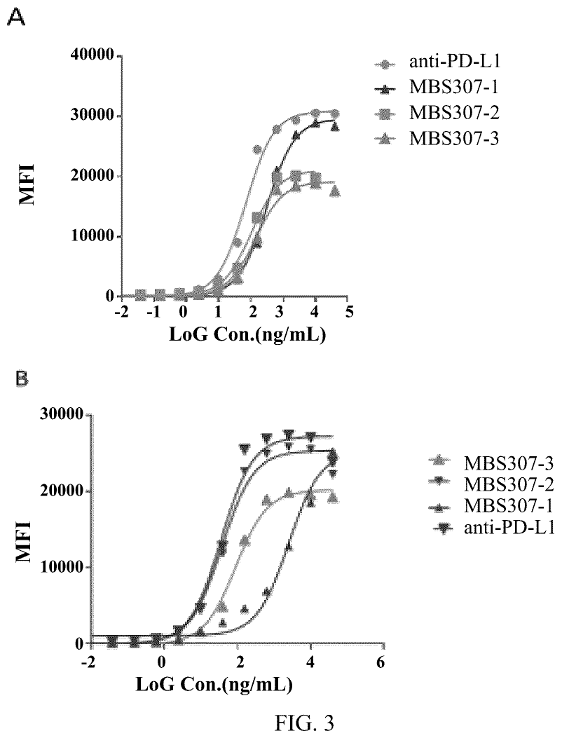

As shown in FIG. 3, all bispecific antibodies bound human and monkey PD-L1s, with the human PD-L1 binding capability similar to that of the monospecific anti-PD-L1 antibody, wherein MBS307-1 had significantly lower binding activity to monkey PD-L1 than the monospecific anti-PD-L1 antibody.

### Example 6. Effect of Bispecific Antibodies on Dendritic Cell Maturation

The effect of the bispecific antibodies on dendritic cell maturation was assayed. Briefly. PBMCs from healthy human donors' blood samples were collected by density gradient centrifugation, suspended in RPM11640 medium, and cultured at 37° C. for 2 h. The adherent cells, i.e., the monocytes, were harvested, and cultured in RPM11640 medium with 100 ng/ml recombinant human GM-CSF (Cat #:7954-GM. R&D, USA), 100 ng/nil recombinant human IL-4 (Cat #:6507-IL, R&D, USA) and 10% FBS. Three days later, half of the medium was replaced with the fresh one. On the 6^{th} day of cell culture, part of the cells were collected and subjected to PD-L1 staining and FACS to check PD-L1 expression. Then, 10¹ monocytes in 100 μl RPMI1640 medium containing 100 ng/ml recombinant human GM-CSF (Cat #:7954-GM. R&D, USA), 100 ng/mi recombinant human IL-4 (Cat #:6507-IL, R&D, USA) and 10% FBS were added with 50 μl bispecific antibodies of the disclosure, the anti-PD-L1 antibody 56E5VH5VL4, the anti-CD40 antibody 7B4VH2VL2 and an anti-Hel antibody as the negative control at different concentrations respectively, and cultured for another 48 h. The cells were stained for the dendritic cell activation markers with the mouse anti-human CD83 antibody (Cat #:556910, BD, USA), PE-mouse anti-human CD86 antibody (Cat #:555658. BD, USA) and BV650 mouse anti-human CD80 antibody (Cat #:564158, BD, USA), and subjected to FACS.

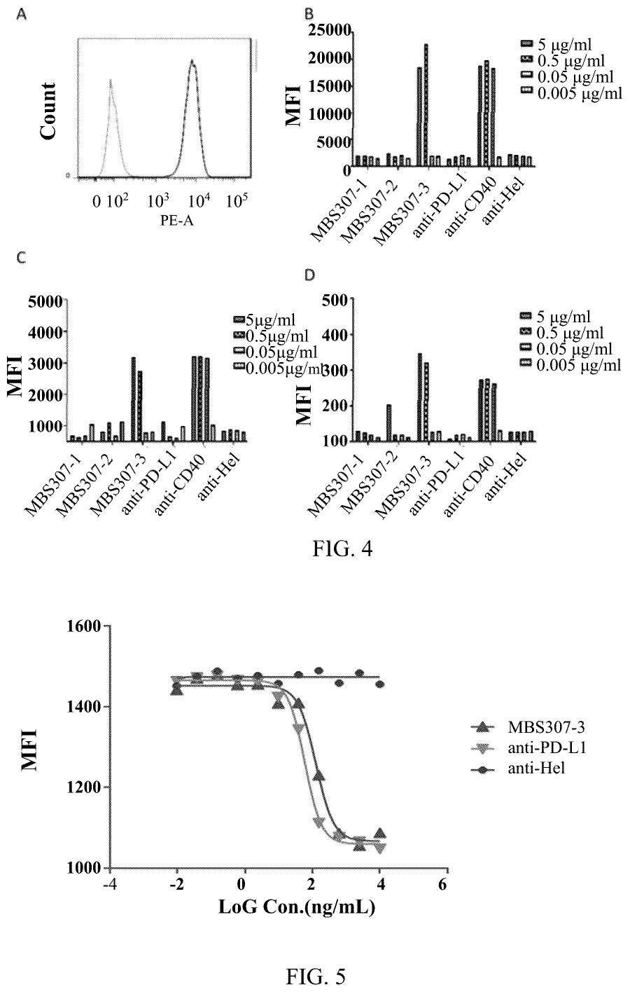

The results were shown in FIG. 4. According to FIG. 4 (A), the dendritic cells were evidently expressing PD-L1 molecules. According to FIG. 4 (B-D), relative to the anti-Hel control, the anti-CD40 antibody and MBS307-3 significantly increased the expression levels of CD86 (a marker for mature dendritic cells), CD80 and CD83 (both are co-stimulatory molecules), while MBS307-1. MBS307-2 and the anti-PD-L1 antibody almost did not show such activity.

### Example 7. Effect of Bispecific Antibodies on PD-L1-PD-1 Binding

The bispecific antibodies were tested for their blocking activity on PD-1-PD-L1 binding by FACS using the HEK293A cells stably expressing human PD-L1 molecules as prepared in Example 1. Briefly, a 96-well plate was seeded with 10⁵ HEK293A/human PD-L1 cells in 100 μl cell culture medium, and then added with 50 μl serially diluted anti-PD-L1 antibody 56E5VH5VL4 or MBS307-3 molecules. After incubation at 4° C. for 1 h. the plate was washed with PBST for 3 times, added with 100 μl 200 μg/ml PD-1-Fc fusion proteins (Cat #:10377-10211, Sino Biological, CN), incubated at 4° C. for 1 h, washed with PBST for 3 times, and added with 1:500 diluted PE-goat anti-human IgG (Cat #:PAI-86078, Thermofisher, USA). After incubation at 4° C. for 1 h, the plate was washed with PBST for 3 times and measured for fluorescence using a FACS machine (BD).

As shown in FIG. 5, 56E5VH5VL4, the monospecific anti-PD-L1 antibody, and the bispecific antibody MBS307-3 both significantly inhibited PD-1-PD-L1, binding, with MBS307-3's blocking capability slightly lower than 56E5VH5VL4.

### Example 8. Effect of Bispecific Antibodies on T Cell Activation

The effect of the antibodies on APC-mediated T cell activation was tested by the mixed lymphocyte reaction (MLR).

Briefly, PBMCs from a healthy human donor's blood sample were collected by density gradient centrifugation, suspended in RPM11640 medium, and cultured at 37° C. for 2 h. The adherent cells, i.e., the monocytes, were harvested, and cultured in 150 μl RPMI1640 medium with 100 ng/ml recombinant human GM-CSF (Cat #:7954-GM, R&D, USA), 100 ng/ml recombinant human IL-4 (Cat #:6507-IL, R&D, USA) and 10% FBS. Three days later, half of the medium was replaced with the fresh one. On the 6^{th} day of cell culture, the cells were added with 50 μl anti-PD-L1 antibody 56E5VH5CL4 (0.01-10 μg/ml), anti-CD40 antibody 7B4VH2VL2 (0.01-10 μg/ml), anti-PD-L1 antibody 56E5VH5CL4+anti-CD40 antibody 7B4VH2VL2 (0.01-10 μg/ml+0.01−10 μg/ml), MBS307-3 (0.01-10 μg/ml) or anti-Hel control (Cat #:LT12031, LifeTein, USA), and cultured for another 48 h. PBMCs from another healthy human donor's blood sample were collected by density gradient centrifugation and suspended in RPMI1640 medium, and CD4⁺ T cells were isolated from the PBMCs using Invitrogen Dynabeads Untouched Human CD4⁺ T cell isolation kit (Cat #:11346D, Thermal Fisher Scientific, USA) according to the manufacturer's instruction. The dendritic cells from the first donor and the CD⁺ T cells from the second donor were plated onto a 96-well U-shape plate at the cell density of 2.5×10⁴ per well and 5×10⁴ per well, respectively, with 150 μl culture medium in total. The plate was added with 50 μl anti-PD-L1 antibody 56E35VH5CL4 (0.01-10 μg/ml), anti-CD40 antibody 7B4VH2VL2 (0.01-10 μg/ml), anti-PD-L1 antibody 56E5VH5CL4+anti-CD40 antibody 7B4VH2VL2 (0.01-10 μg/ml+0.01-10 μg/ml), MBS307-3 (0.01-10 μg/ml) or anti-Hel control (Cat #:LT12031, LifeTein, USA), and incubated for 72 h. The IFN-γ levels were tested by ELISA using a kit (Cat #:SIF50. R&D, USA) according to the manufacturer's instruction.

Alternatively. PBMCs from a healthy human donor's blood sample were collected by density gradient centrifugation, suspended in RPMI1640 medium, and cultured at 37° C. for 2 h. The adherent cells, i.e., the monocytes, were harvested, and cultured in 150 μl RPMI1640 medium with 100 ng/ml recombinant human GM-CSF (Cat #:7954-GM. R&D, USA), 100 ng/ml recombinant human 11-4 (Cat #:6507-IL, R&D, USA) and 10% FBS. Three days later, half of the medium was replaced with the fresh one. On the 6^{th} day of cell culture, the culture medium was replaced with the medium containing 100 ng/ml recombinant human GM-CSF, 100 ng/ml recombinant human IL-4, 10 ng/ml rhTNF-α (Cat #:210-TA-100, R&D, US), 1000 U/ml rhIL-6 (Cat #:7270-IL-025, R&D, US), 1 μg/ml PGE2 (Cat #:363-24-6. TOCRIS, US) and 10 ng/ml IL-1β (Cat #:210-LB-025, R&D, US), and the cells were cultured for another 2 days. PBMCs from another healthy human donor's blood sample were collected by density gradient centrifugation and suspended in RPMI1640 medium, and CD4⁺ T cells were isolated from the PBMCs using Invitrogen Dynabeads Untouched Human CD4⁺ T cell isolation kit (Cat #:11346D, Thermal Fisher Scientific, USA) according to the manufacturer's instruction. The dendritic cells from the first donor and the CD4⁺ T cells from the second donor were plated onto a 96-well U-shape plate at the cell density of 2.5×10⁴ per well and 5×10⁴ per well, with 150 μl culture medium in total. The plate was added with 50 μl anti-PD-L1 antibody 56E5VH5CL4 (0.01-10 μg/ml), anti-CD40 antibody 7B4VH2VL2 (0.01-10 μg/ml), anti-PD-L1 antibody 56E5VH5CL4+anti-CD40 antibody 7B4VH2VL2 (0.01-10 μg/ml+0.01-10 μg/ml), MBS307-3 (0.01-10 μg/ml) or anti-Hel control (Cat #:LT12031, LifeTein, USA), and incubated for 72 h. The IFN-γ levels were tested by ELISA using a kit (Cat #:SIF50, R&D, USA) according to the manufacturer's instruction. The assay was done in triplicate.

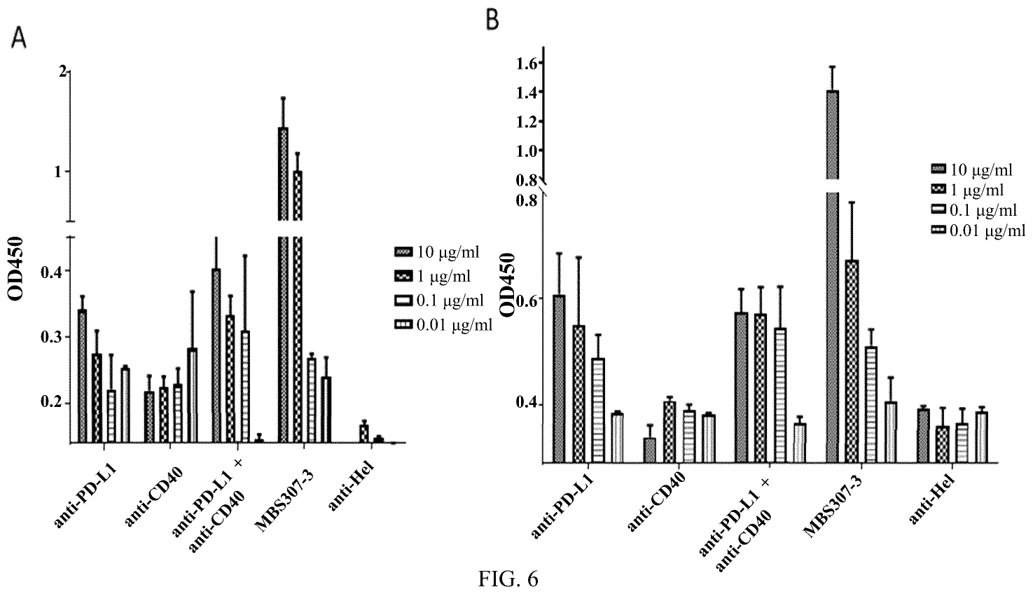

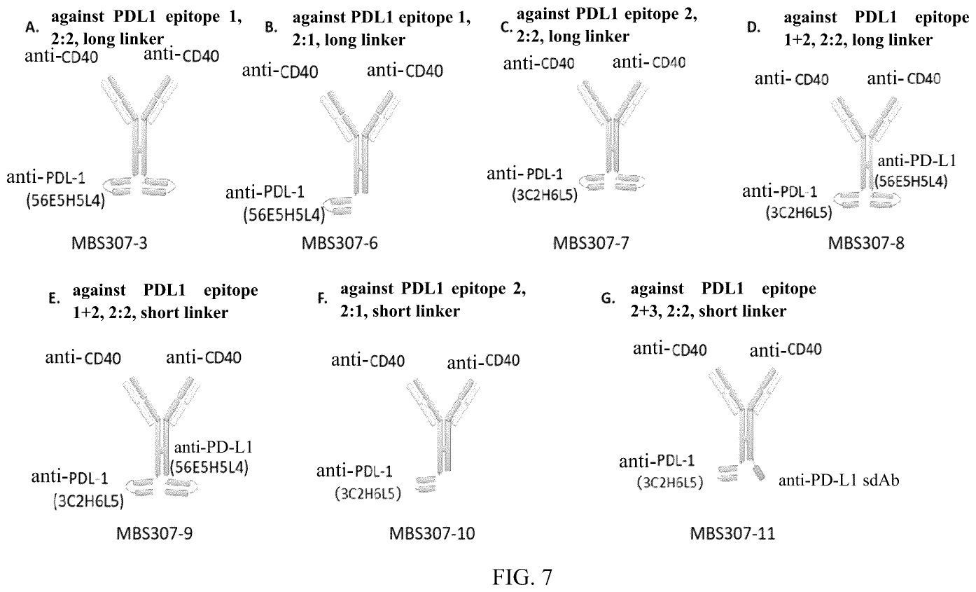

The results were shown in FIG. 6. According to FIG. 6 (A), the anti-PD-L1 antibody and the anti-CD40 antibody showed higher activity in inducing T cell activation if they were used at both the dendritic cell maturation stage and the T cell activation stage, and the use of the combination of these two antibodies or MBS307-3 induced T cell activation at an even higher activity as compared to the monospecific anti-PD-L1 or anti-CD40 antibody. According to FIG. 6 (B), if the dendritic cell maturation was induced by cytokines only, then the anti-CD40 antibody showed lower activity in T cell activation, while the activity of the anti-PD-L1 antibody, and the combination of the two monospecific antibodies or MBS307-3 remained unchanged, as compared to the results shown in FIG. 6 (A).

### Example 9. Construction. Expression and Assembly of Bispecific Antibodies with Optimized Structures

The bispecific antibodies of the disclosure were optimized in structure according to FIG. 7, to obtain new bi- or multi-specific antibodies.

The CD40 binding domain still used a heavy chain variable region and a light chain variable region comprising the amino acid sequences of SEQ ID NOs: 1 and 2, respectively, while the PD-L1 binding domain further used a heavy chain variable region and a light chain variable region comprising the amino acid sequences of SEQ ID NOs: 10 and 11, respectively (i.e., the heavy and light chain variable regions of the anti-PD-L1 antibody 3C2VH6VL5), and an anti-PD-L1 nanobody of SEQ ID NO: 52, in addition to the heavy and light chain variable regions of SEQ ID NOs: 3 and 4.

Two half antibodies MBS307-6-1 (comprising the polypeptide chain of SEQ ID NO: 12 containing anti-CD40 heavy chain variable region-knob variant of heavy chain constant region, the polypeptide chain of SEQ ID NO: 53 containing anti-CD40 light chain variable region-light chain constant region) and MBS307-6-2 (comprising the polypeptide chain of SEQ ID NO: 13 containing anti-CD40 heavy chain variable region-hole variant of heavy chain constant region-linker 3-anti-PD-L1 heavy chain variable region (56E5VH5VL4)-linker 1-anti-PD-L1 light chain variable region (56E5VH5VL4), the polypeptide chain of SEQ ID NO: 53 containing the anti-CD40 light chain variable region-light chain constant region) for the asymmetrical anti-CD40/PD-L1 antibody MBS307-6; the full-length antibody MBS307-7 (comprising the polypeptide chain of SEQ ID NO: 18 containing anti-CD40 heavy chain variable region-heavy chain constant region-linker 3-anti-PD-L1 heavy chain variable region (3C2VH6VL5)-linker 1-anti-PD-L1 light chain variable region (3C2V_{H}6VL5), the polypeptide chain of SEQ ID NO: 53 containing the anti-CD40 light chain variable region-light chain constant region); two half antibodies MBS307-8-1 (comprising the polypeptide chain of SEQ ID NO: 14 containing anti-CD40 heavy chain variable region-knob variant of heavy chain constant region-linker 3-anti-PD-L1 heavy chain variable region (3C2VH6VL5)-linker 1-anti-PD-IL light chain variable region (3C2VH6VL5), the polypeptide chain of SEQ ID NO: 53 containing the anti-CD40 light chain variable region-light chain constant region) and MBS307-8-2 (comprising the polypeptide chain of SEQ ID NO: 13 containing anti-CD40 heavy chain variable region-hole variant of heavy chain constant region-linker 3-anti-PD-L1, heavy chain variable region (56E5VH5VL4)-linker 1-anti-PD-L1 light chain variable region (56EV5VL4), the polypeptide chain of SEQ ID NO: 53 containing the anti-CD40 light chain variable region-light chain constant region) for the asymmetrical anti-CD40/PD-L1 antibody MBS307-8: two half antibodies MBS307-9-1 (comprising the polypeptide chain of SEQ ID NO: 15 containing anti-CD40 heavy chain variable region-knob variant of heavy chain constant region-linker 4-anti-PD-L1 heavy chain variable region (56E5VH5VL4)-linker 1-anti-PD-L1 light chain variable region (56E5VH5VL4), the polypeptide chain of SEQ ID NO: 53 containing the anti-CD40 light chain variable region-light chain constant region) and MBS307-9-2 (comprising the polypeptide chain of SEQ ID NO: 16 containing anti-CD40 heavy chain variable region-hole variant of heavy chain constant region-linker 4-anti-PD-L1 heavy chain variable region (3C2VH6VL5)-linker 1-anti-PD-L1 light chain variable region (3C2VH6VL5), the polypeptide chain of SEQ ID NO: 53 containing the anti-CD40 light chain variable region-light chain constant region) for the asymmetrical anti-CD40/PD-L1 antibody MBS307-9; two half antibodies MBS307-10-1 (comprising the polypeptide chain of SEQ ID NO: 12 containing anti-CD40 heavy chain variable region-knob variant of heavy chain constant region, the polypeptide chain of SEQ ID NO: 53 containing the anti-CD340 light chain variable region-light chain constant region) and MBS307-10-2 (comprising the polypeptide chain of SEQ ID NO: 16 containing anti-CD40 heavy chain variable region-hole variant of heavy chain constant region-linker 4-anti-PD-L1 heavy chain variable region (3C2VH6VL5)-linker 1-anti-PD-L1 light chain variable region (3C2V H6VL5), the polypeptide chain of SEQ ID NO: 53 containing the anti-CD40 light chain variable region-light chain constant region) for the asymmetrical anti-CD40/P1D-L1 antibody MBS307-10; two half antibodies MBS307-11-1 (comprising the polypeptide chain of SEQ ID NO: 17 containing anti-CD40 heavy chain variable region-knob variant of heavy chain constant region-linker 4-anti-PD-L1 nanobody, the polypeptide chain of SEQ ID NO: 53 containing the anti-CD40 light chain variable region-light chain constant region) and MBS307-11-2 (comprising the polypeptide chain of SEQ ID NO: 16 containing anti-CD40 heavy chain variable region-hole variant of heavy chain constant region-linker 4-anti-PD-L1 heavy chain variable region (3C2VH6VL5)-linker 1-anti-PD-L1 light chain variable region (3C2VH6VL5), the polypeptide chain of SEQ ID NO: 53 containing the anti-CD40 light chain variable region-light chain constant region) for the asymmetrical anti-CD40/PD-L1 antibody MBS307-11 were constructed. The antibody constituents were shown in FIG. 7, Table 1 and the sequence listing.

TABLE 1
Long chains of bi- or multi-specific antibodies

| | | SEQ | | | Knob-half | Hole-half |
|---|---|---|---|---|---|---|
| | | ID | | | antibody | antibody |
| Structure | Ab ID | NO. | Structure | Ab ID | SEQ ID NO. | SEQ ID NO. |
| symmetric | MBS307-2 | 8 | asymmetric | MBS307-1 | 5 | 7 |
| | MBS307-3 | 9 | | MBS307-6 | 12 | 13 |
| | MBS307-7 | 18 | | MBS307-8 | 14 | 13 |
| | | | | MBS307-9 | 15 | 16 |
| | | | | MBS307-10 | 12 | 16 |
| | | | | MBS307-11 | 17 | 16 |

The DNA fragment synthesis, expression vector construction, cell transfection, and antibody purification were done following the protocol in Example 2, and the asymmetric antibodies were assembled according to the protocol in Example 3.

### Example 10. Binding Capability of Bi- or Multi-Specific Antibodies to Human CD40 and Human PD-L1

Following the protocols in Example 4 and Example 5, the bi- or multi-specific antibodies of the disclosure were tested for their binding capability to human PD-L1 and human CD40 proteins by FACS using the HEK293A cells prepared in Example 1 that stably expressed human PD-L1 or human CD40.

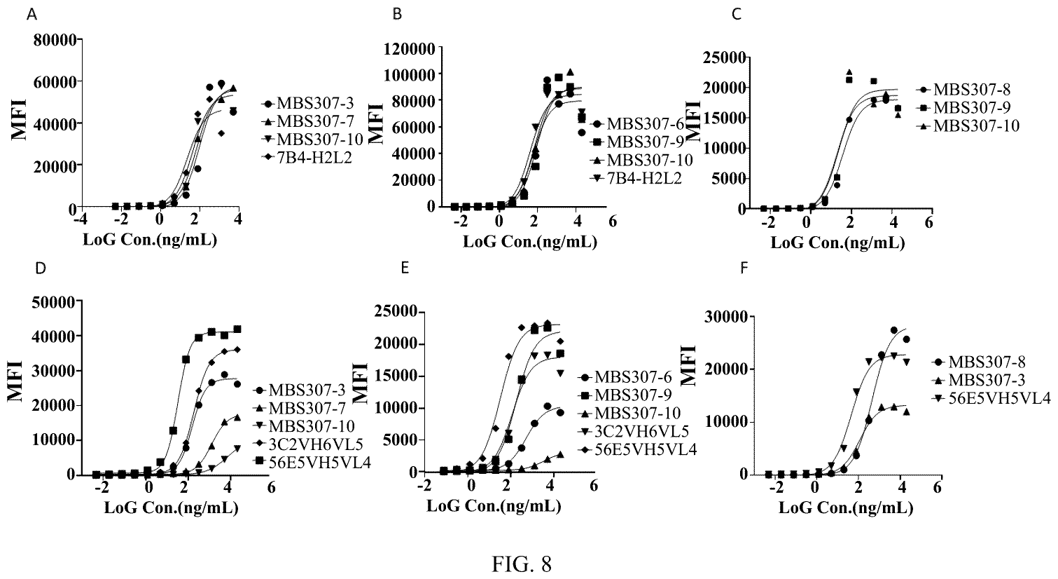

The results were shown in FIG. 8, MBS307-3. MBS307-6, MBS307-7, MBS307-8, MBS307-9 and MBS307-10 had similar binding capability to human CD40 (FIG. 8 (A-C)) but different human PD-L1 binding capability (FIG. 8 (D-F)), wherein MBS307-10 showed lowest PD-L1 binding capability while MBS307-8 and MBS307-9 showed highest human PD-L1 binding activity.

### Example 11. Effect of Bi- or Multi-Specific Antibodies on CD40 Signaling Activation

HEK-Blue™ Null I_v cells (InvivoGen, USA) were transfected with lentiviruses expressing human CD40 molecules (SEQ ID NO: 25) as prepared following the protocol of Example 1, to obtain a HEK-Blue reporter cell line.

The bi- or multi-specific antibodies were tested for their capability to activate the CD40 signaling using the HEK-Blue reporter cells. Briefly, the HEK-Blue/CD40 cells were incubated in DMEM medium (Cat #:SH30243.01, Hyclone, USA) containing 10% FBS (Cat #: FND500, Excell, CN), 10 μg/ml puromycin (Cat #:A11138-03, GIBCO, USA), 100 μg/ml Normocin™ (Cat #:ant-nr-2, Invivogen, USA), and 100 μg/ml bleomycin (Cat #:ant-Zn-5, Invivogen, USA). The cell culture plate was coated with 50 μl 2 μg/ml human PD-L1-his (Cat #:10084-H08H-100, Sino Biological, CN) overnight (16 h), added with 100 μl HEK Blue™ detection buffer (Cat #:hb-det3, Invivogen. USA) containing 4×10⁴ HEK-Blue/CD40 cells and the bi- or multi-specific antibodies or the anti-CD40 monospecific antibody 7B4VH2VL2 at different concentrations (from 100 μg/ml to 0.01 ng/ml), and incubated at 37° C. overnight to have blue color developed (˜24 h). The plate was read for OD630 using the SpectraMaxR i3X reader (Molecular Devices, USA).

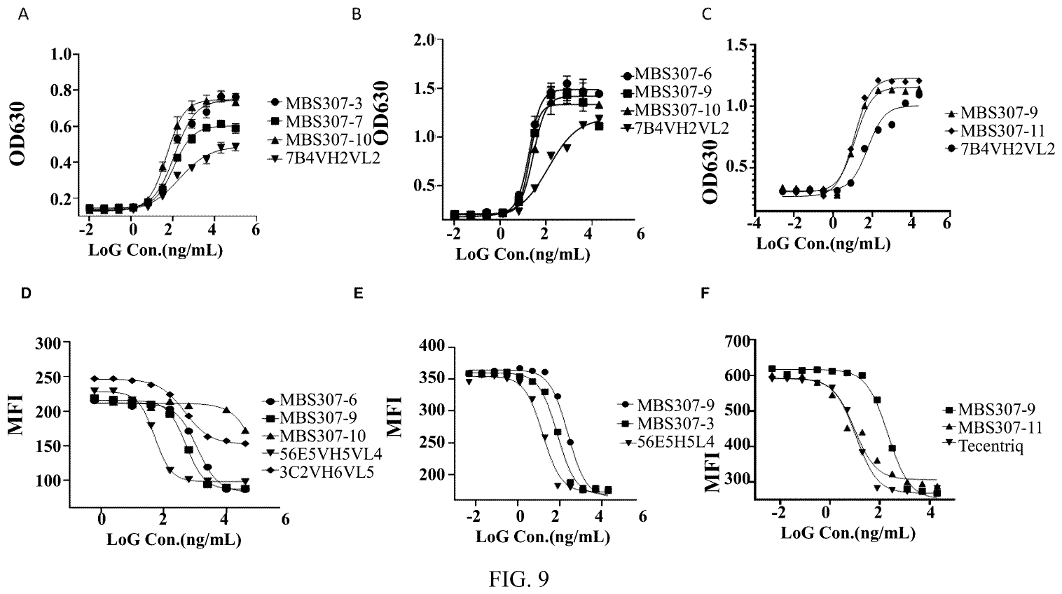

The results were shown in FIG. 9 (A-C). All bi- or multi-specific antibodies showed higher activity in CD40 signaling activation than the monospecific anti-CD40 antibody 7B4VH2VL2. Among these bi- or multi-specific antibodies, MBS307-6, MBS307-9, MBS307-10 and MBS307-11 had comparable but highest activity, which was higher than that of MBS307-3 and MBS307-7.

### Example 12. Blocking Capability of Bi- or Multi-Specific Antibodies on PD-1-PD-L1 Interaction

Following the protocol of Example 7, the bi- or multi-specific antibodies were tested for their capability to block PD-1-PD-L1 binding by FACS, using the HEK293A cells prepared in Example 1 that stably expressed human PD-L1 molecules.

The results were shown in FIG. 9 (D-F). The bi- or multi-specific antibodies showed quite different blocking capability on PD-1-PD-L11 binding, with MBS307-10 having the lowest capability, which was much lower than that of 56E5VH5VL4 and 3C2VH6VL5, two monospecific anti-PD-L1 antibodies, and other bi- or multi-specific antibodies of the disclosure. MBS307-11 exhibited the highest blocking activity, which was higher than that of 3C2VH6VL5 and other bi- or multi-specific antibodies of the disclosure, and comparable to that of Tecentriq, a monospecific anti-PD-L1 antibody.

### Example 13. Effect of Bi- or Multi-Specific Antibodies on Dendritic Cell Maturation and T Cell Activation

Following the protocol of Example 6, the bi- or multi-specific antibodies were tested for their capability on dendritic cell maturation.

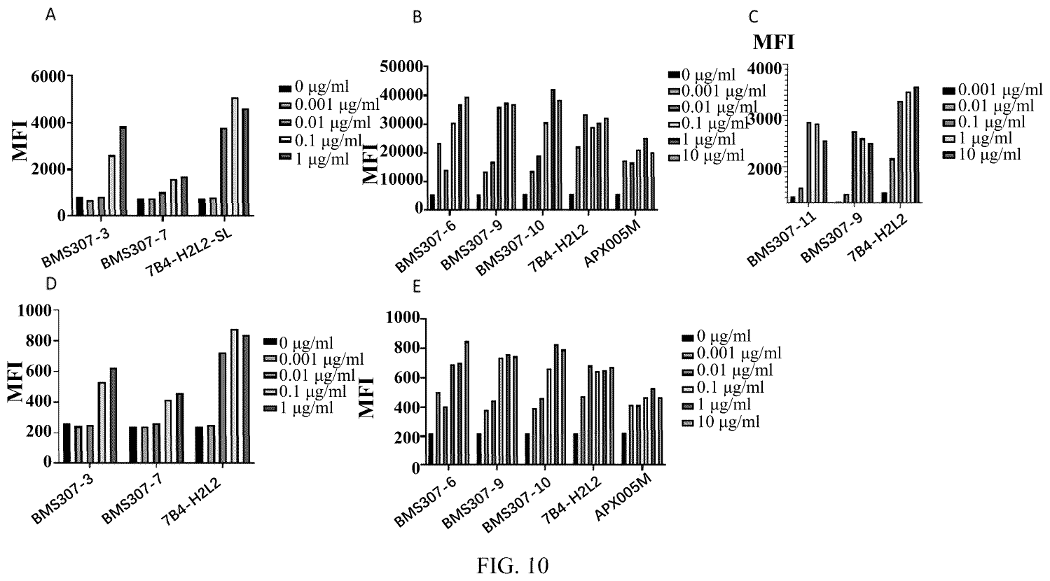

With the assay described in Example 8, the bi- or multi-specific antibodies were tested for their effect on T cell activity. Briefly, PBMCs from a healthy human donor's blood sample were collected by density gradient centrifugation, suspended in RPM11640 medium, and cultured in an incubator at 37° C. for 2 h. The adherent cells, i.e., the monocytes, were harvested, and cultured in 150 μl RPMI1640 medium with 100 ng/ml recombinant human GM-CSF (Cat #:7954-GM, R&D, USA), 100 ng/ml recombinant human 1L-4 (Cat #:6507-IL, R&D, USA) and 10% FBS. Three days later, half of the medium was replaced with the fresh one. On the 6^{th} day of cell culture, the cells were added with 50 μl 7B4VH2VL2 (an anti-CD40 antibody, 0.01-10 μg/ml), APX005M (an anti-CD40 antibody, 0.01-10 μg/ml), MBS307-6 (0.01-10 μg/ml), MBS307-9 (0.01-10 μg/ml), and MBS307-10 (0.01-10 μg/ml), respectively, and cultured for another 48 h. PBMCs from another healthy human donor's blood sample were collected by density gradient centrifugation and suspended in RPMI1640 medium, and CD4⁺ T cells were isolated from the PBMCs using Invitrogen Dynabeads Untouched Human CD4⁺ T cell isolation kit (Cat #: 11346D, Thermal Fisher Scientific, USA). The dendritic cells from the first donor and the CD4⁺ T cells from the second donor were plated onto a 96-well U-shape plate at the cell density of 2.5×10⁴ per well and 5×10⁴ per well, with 150 μl culture medium in total. The plate was added with 50 μl 7B4VH2VL2, APX005M, MBS307-6, MBS307-9, and MBS307-10, at different concentrations (see FIG. 10 for the detailed concentrations), respectively, and incubated for 72 h. The IFN-7, IL-6 and IL-2 levels were tested by ELISA using the kits (Cat #:SIF50, R&D, USA; Cat #:D6050, R&D, USA; Cat #:D2050, R&D, USA) according to the manufacturers' instruction.

The results were shown in FIG. 10. According to the expression levels of CD86 and CD83, the two markers for DC maturation. MBS307-6, MBS307-9, MBS307-10 and MBS307-11 showed the highest capability of promoting DC maturation, which was comparable to that of 7B4VH2VL2.

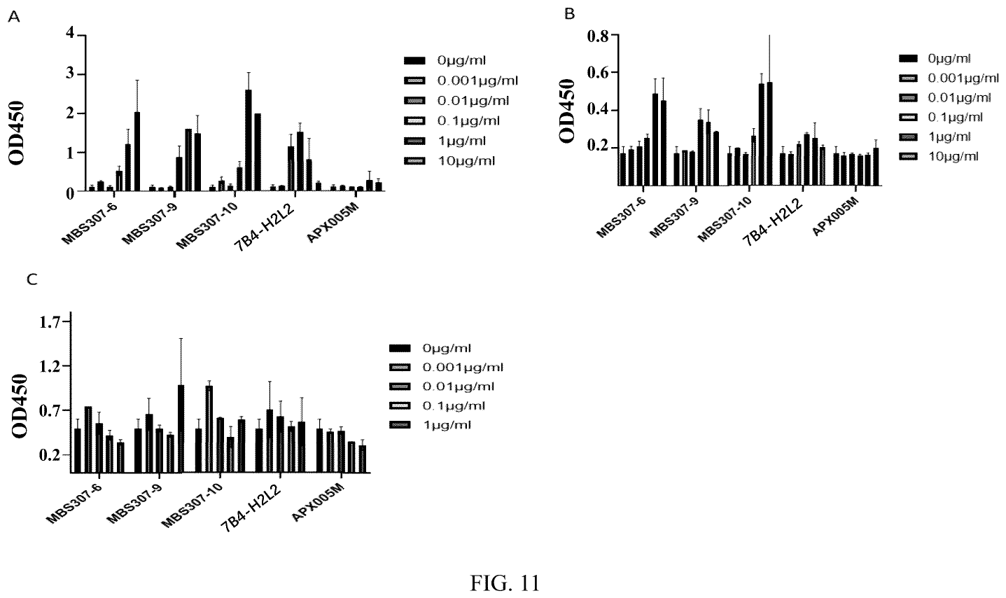

According to FIG. 11, the bi- or multi-specific antibodies, including MBS307-6, MBS307-9 and MBS307-10, significantly induced cytokine release by T cells, with the activity much higher than that of APX005M and comparable to or a bit higher than that of 7B4V H2VL2.

The bi- or multi-specific antibodies of the disclosure were further tested for their effect on T cell activation when used in combination with Tecentriq, the monospecific anti-PD-L1 antibody developed by Roche, following the protocol above except that T cells were treated with the bi- or multi-specific antibodies and 0.1 μg/ml Tecentriq in combination.

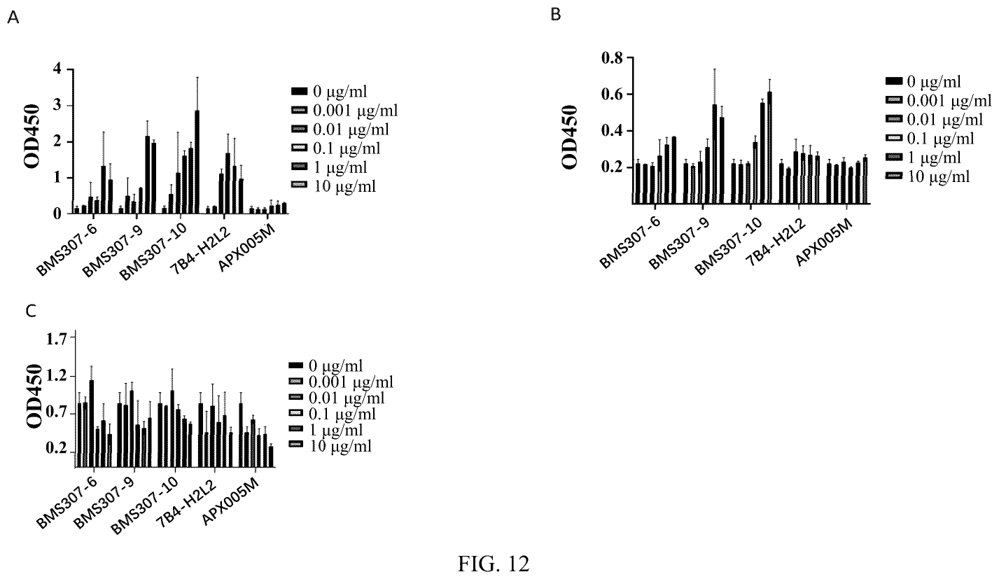

According to FIG. 12, the combined use of the bi- or multi-specific antibodies, including MBS307-6, MBS307-9 and MBS307-10, and 0.1 μg/mi Tecentriq, activated T cells in a concentration dependent manner, as revealed by the increased levels of IFN-γ and IL-6, suggesting their synergistic effect on immune system activation, and the synergistic effect was higher than that of the combination of APX005M and 0.1 μg/ml Tecentriq, and comparable to or a bit higher than that of the combination of 7B4VH2VL2 and 0.1 μg/ml Tecentriq.

In another assay, the T cells were treated with Tecentriq at different concentrations (0-5 μg/ml) and the bi- or multi-specific antibodies, 7B4VH2VL2 or APX005M at a fixed concentration (1 μg/ml), and subjected to IFN-γ and IL-2 level determination using the two kits (Cat #:SIF50, R&D, USA; Cat #:D2050, R&D, USA) according to the manufacturers' instruction.

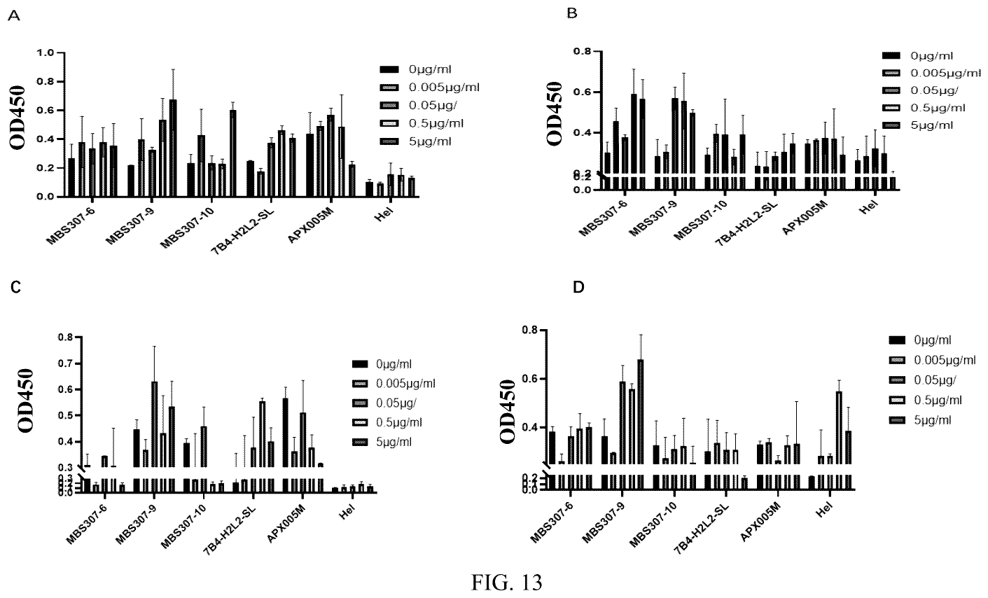

As shown in FIG. 13 (A-B), the combination of Tecentriq at different concentrations with the bi- or multi-specific antibodies, especially MBS307-6 and MBS307-9, promoted T cell activation, increasing IL-2 secretion, indicating their synergistic effect on immune system activation, and their synergistic effect was comparable to or a bit higher than the combination of Tecentriq at different concentrations with 7B4VH2VL2 or APX005M.

In another assay performed in parallel, the T cells were treated with Keytruda, an anti-PD-L1 monospecific antibody, at different concentrations (0-5 μg/ml) and each bi- or multi-specific antibody, 7B4VH2VL2 or APX005M at a fixed concentration (1 μg/ml), and subjected to IFN-γ and IL-2 level determination using the two kits (Cat #:SIF50, R&D, USA; Cat #:D2050, R&D, USA) according to the manufacturers' instruction.

As shown in FIG. 13 (C-D), the combination of Keytruda at different concentrations with MBS307-9, promoted T cell activation, increasing IFN-γ and IL-2 levels, indicating their synergistic effect on immune system activation, and their synergistic effect was comparable to or a bit higher than the combination of Keytruda at different concentrations with 7B4VH2VL2 or APX005M.

### Example 14. Binding Affinity of Bi- or Multi-Specific Antibodies to Human CD40 and PD-L1

Using BIAcore™ 8K instrument (GE Life Sciences, US), the binding affinity of the bi- or multi-specific antibodies to human CD40 and PD-L1 was quantitatively measured. Briefly, 100-200 response units (RU) of human CD40-his protein (Cat #:10774-1108H, Sino Biological, CN) or human PD-L1-his protein (Cat #:10084-H08H, Sino Biological, CN) were coupled to CM5 biosensor chips (Cat #:BR-1005-30, GE Life Sciences. USA). The un-reacted groups were then blocked with 1 M ethanolamine. Serially diluted antibodies at concentrations ranging from 0.3 μM to 10 μM were injected into the SPR running buffer (HBS-EP buffer, pH7.4, Cat #:BR-1006-69, GE Life Sciences, USA) at 30 μL/min. The binding affinity was calculated with the RUs of blank controls subtracted, and the association rate (kₐ) and dissociation rate (k_{d}) were determined using the one-to-one Langmuir binding model (BIA Evaluation Software, GE Life Sciences, USA). The equilibrium dissociation constant K_{D} was calculated as the k_{d}/kₐ ratio.

According to the SPR binding curves, the binding affinity data of the bi- or multi-specific antibodies to human CD40 and PD-L1 proteins were determined and summarized in Table 2. The bi- or multi-specific antibodies with different structures showed comparable binding affinity to human CD40 protein but quite different binding affinity to human PD-L1 protein, with MBS307-11 having the highest PD-L1 binding affinity.

TABLE 2
Binding affinity of bi- or multi-specific antibodies to human CD40 and PD-L1

| | human CD40 | | | human PD-L1 | | |
|---|---|---|---|---|---|---|
| Ab | Kₐ | K_{d} | K_{D} | Kₐ | K_{d} | K_{D} |
| MBS307-6 | 5.46E+06 | 1.70E−02 | 3.11E−09 | 1.72E+05 | 7.22E−03 | 4.20E−08 |
| MBS307-9 | 7.90E+06 | 2.29E−02 | 2.90E−09 | 1.43E+05 | 9.73E−05 | 6.83E−10 |
| MBS307-10 | 7.19E+06 | 1.96E−02 | 2.72E−09 | 9.27E+04 | 1.64E−03 | 1.77E−08 |
| MBS307-3 | 1.25E+07 | 1.77E−02 | 1.42E−09 | 2.12E+05 | 6.80E−05 | 3.21E−10 |
| MBS307-7 | 1.33E+07 | 1.90E−02 | 1.43E−09 | 1.32E+05 | 1.58E−03 | 1.19E−08 |
| MBS307-11 | 3.70E+06 | 1.62E−02 | 4.37E−09 | 1.02E+06 | 7.07E−05 | 6.90E−11 |

The bi- or multi-specific antibodies of the disclosure were further tested by SPR for their capability to bind two antigens simultaneously, using anti-human Fe biosensor chips. In particular, 1 μg/ml MBS307-6, MBS307-9 and MBS307-10 were respectively coupled to a CM5 biosensor chip (Cat #: 10266084, GE Life Sciences, USA). Serially diluted CD40 molecules (2-fold dilution starting at 4 μg/ml, 8 concentrations in total) and serially diluted PD-L1 molecules (2-fold dilution starting at 4 μg/ml) were injected into the SPR running buffer (HBS-EP buffer, pH7.4, Cat #:BR-1006-69, GE Life Sciences. USA) in said order or the reverse order at 30 μL/min. The first antigen-antibody association kinetics was followed for 180 s and the dissociation kinetics was followed for 500 s. The second antigen-antibody association kinetics was followed for 180 s and the dissociation kinetics was followed for no less than 500 s. The binding affinity was calculated with the RUs of blank controls subtracted.

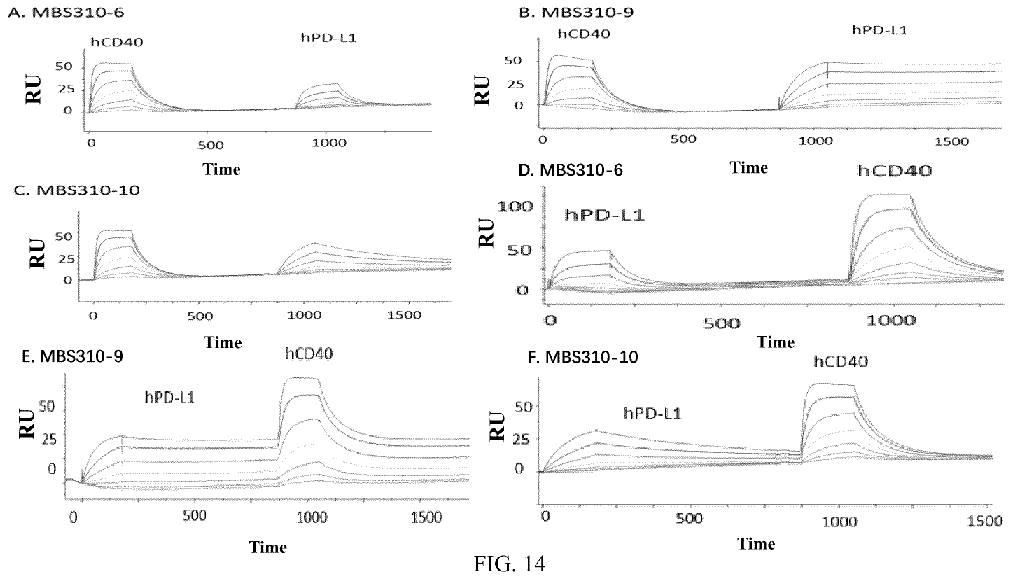

The results were shown in FIG. 14(A-C, CD40 injection followed by PD-L1 injection; D-F. PD-L1 injection followed by CD40 injection). MBS307-6. MBS307-9 and MBS307-10 were able to bind CD40 and PD-L1 molecules simultaneously, no matter in which order they were exposed to the two antigens, and the kinetics data was quite consistent to those obtained when the binding affinity to single antigens was measured.

### Example 15. Epitope Binning

To further determine the epitopes the two binding arms of the bi- or multi-specific antibodies bind, a competitive SPR binding assay was performed. Briefly, 1 μg/ml human PD-L1 (ECD)-his proteins (Cat #:10084-H08H, Sino Biological, CN) were coupled to a CM5 biosensor chip (Cat #:BR-1005-30, GE Life Sciences, USA). The un-reacted groups were then blocked with 1 M ethanolamine. The anti-PD-L1 antibodies, 3C2VH6VL5 and 56E5VH5VL4, of 5 μg/ml were respectively injected into the SPR running buffer (IBS-EP buffer, pH7.4, Cat #:BR-1006-69, GE Life Sciences, USA) at 30 μl/min. Then, a second anti-PD-L 1 antibody (i.e., the anti-PD-L1 nanobody) of 5 μg/ml was injected into the SPR running buffer at 30 μL/min. The binding affinity was calculated with the RUs of blank controls subtracted, and the association rate (kₐ) and dissociation rate (k_{d}) were determined using the one-to-one Langmuir binding model.

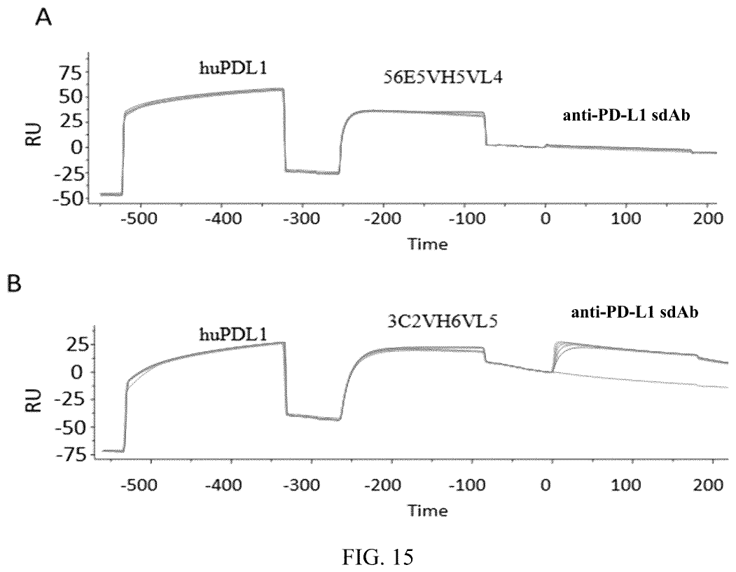

The results were shown in FIG. 15. Particularly, 3C2VH6VL5 and the anti-PD-L1 nanobody bound to the PD-L1 molecules simultaneously, indicating they bound human PD-L1 at different epitopes without competition, while 56E5VH5VL4 and the anti-PD-L1 nanobody did not bind to the PD-L1 antigen simultaneously, suggesting they bound at the same, similar or overlapping epitope(s), with competition to some extent.

With the same protocol, the competitive PD-L1 epitope binding was tested among 3C2VH6VL5, the anti-PD-L1 nanobody, and MBS307-11. In particular, 3C2VH6VL5, the anti-PD-L1 nanobody, and MBS307-11 of 5 μg/ml were respectively injected into the SPR running buffer (HBS-EP buffer, pH7.4, Cat #:BR-1006-69, GE Life Sciences, USA) at 30 μL/min. Then, a second anti-PD-L1 antibody. i.e., 3C2VH6VL5, the anti-PD-L1 nanobody, or MBS307-11, of 5 μg/ml was injected into the SPR running buffer at 30 μL/min.

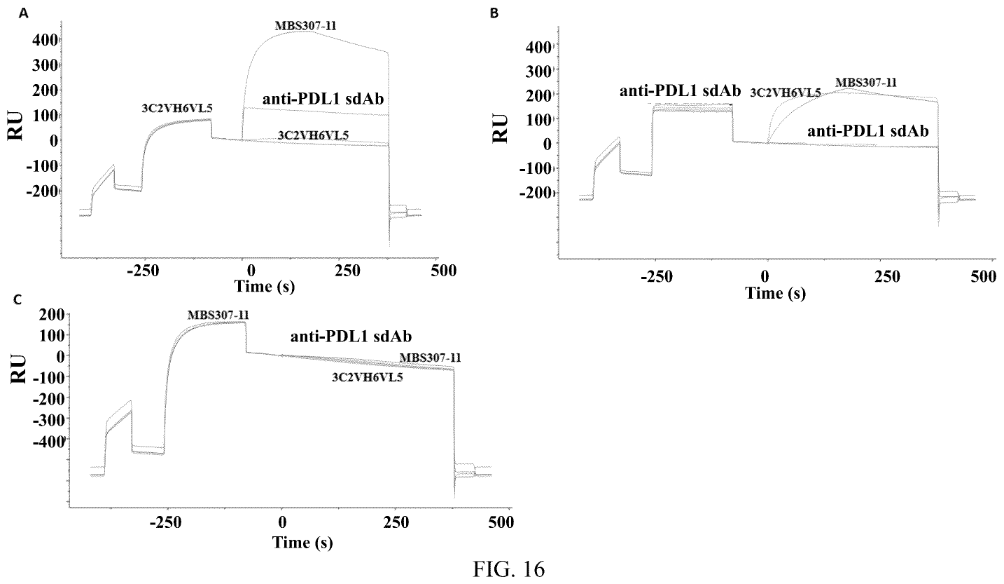

The results were shown in FIG. 16. In consistent with the result above, 3C2VH6VL5 and the anti-PD-L1, nanobody bound to the PD-L1 molecules simultaneously. The epitope to which MBS307-11 bound may fully or partially overlap that bound by 3C2VH46VL5 or the anti-PD-L1 nanobody, as with the binding of MBS307-11, 3C2VH6VL5 and the anti-PD-L1 nanobody were not able to bind to the PD-L1 molecules.

### Example 16. In Vivo Anti-Tumor Efficacy of Bi- or Multi-Specific Antibodies

The in vivo anti-tumor activity of MBS307-11, 7B4VH2VL2-mFc (an anti-CD40 monospecific antibody, containing 7B4VH2VL2's heavy and light chain variable regions and mouse IgG1/κ constant regions of SEQ ID NOs: 45 and 54, respectively), Tecentriq (an anti-PD-L1 monospecific antibody), and the combination of 7B4VH2VL2-mFc and Tecentriq was tested, using an animal model generated by transplanting transgenic mice carrying functional human CD40, PD-1 and PD-L1 genes (Biocytogen, CN) with MC38-hPD-L1 cells, wherein the MC38-hPD-L1 cells were prepared by engineering the MC38 murine colon carcinoma cells with over-expression of human PD-L1 molecules and knock-out of murine PD-L1 gene.

Briefly, 56 transgenic B-hPD-L1/hPD-L1/hCD40 mice were subcutaneously injected with 5×10⁵ MC38-hPD-L1 cells in 0.1 ml medium at the right flank. When the average tumor size reached 100 to 150 mm³, the animals were allocated into 5 groups according to the tumor size and body weight, 8 mice per group. The mice were intraperitoneally administered with MBS307-11 (15 mg/kg), 7B4VH2VL2-mFc (10 mg/kg), Tecentriq (10 mg/kg), 7B4VH2VL2-mFc (10 mg/kg)+Tecentriq (10 mg/kg) (mixed before administration) and PBS, respectively, on Day 0, 4, 7, 11, 14 and 18. Forty-eight hours post first administration, blood samples were collected from the mice and measured for the levels of alanine aminotransferase (ALT) and aspartate aminotransferase (AST) using HITACHI automatic biochemical analyzer 3110. Tumor sizes and mouse body weights were monitored over time. In specific, the tumor size was determined by measuring by a caliper the length (the longest diameter. D) and the width (the diameter perpendicular to the length, d) of a tumor and calculating the volume as 0.5×D×d². The test was terminated before the tumor sizes in the administration groups reached 3.5 cm³. One-way ANOVA was used to identify tumor size differences among groups.

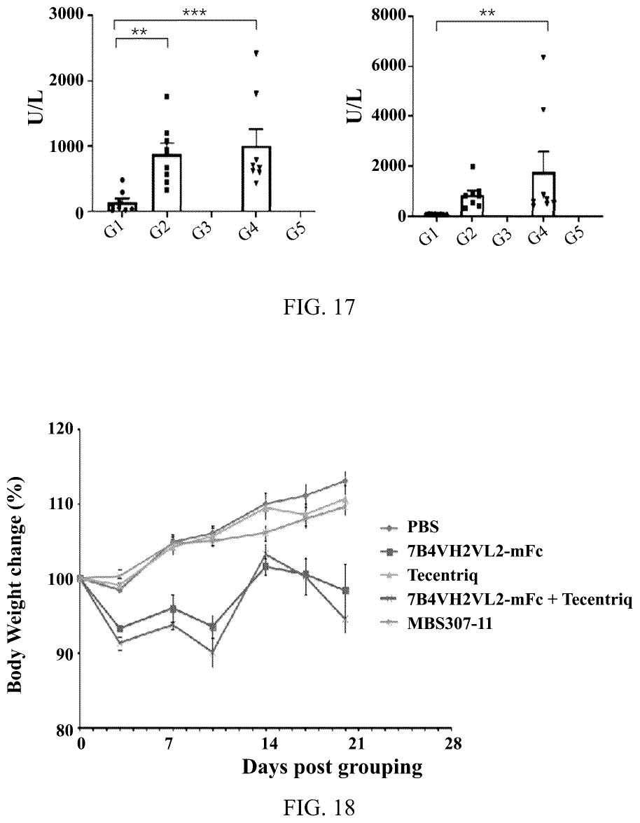

As shown in FIG. 17, the ALT and AST levels in the groups treated with 7B4VH2VL2-mFc or 7B4VH2VL2-mF-+-Tecentriq were significantly higher than those of the vehicle control group, suggesting the agent(s) caused a certain level of hepatotoxicity. However, the ALT and AST levels in the MBS307-11 and Tecentriq administration groups were not different from those of the vehicle control group, indicating the design of the bi- or multi-specific antibodies of the disclosure removed the anti-CD40 antibody-mediated toxicity, causing no hepatotoxicity.

According to FIG. 18, the mice significantly lost weight in the groups treated with 7B4VH2VL2-mFc or 7B4VH2VL2-mFc+Tecentriq, meaning the agent(s) had toxicity, while the mice in the MBS307-11 and Tecentriq administration groups did not lose weight, indicating the design of the bi- or multi-specific antibodies of the disclosure removed the anti-CD340 antibody-mediated toxicity.

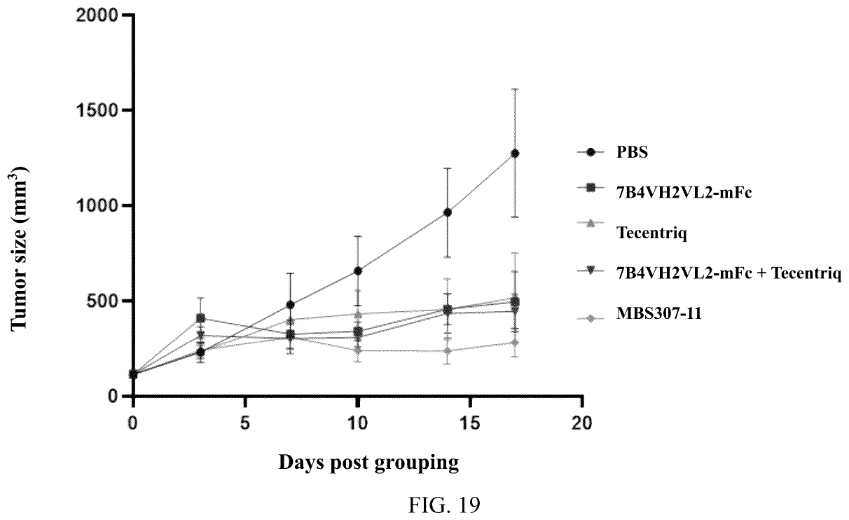

Further, as shown in FIG. 19, the tumor growth was significantly inhibited in the groups treated with the antibodies, wherein the anti-tumor efficacy of the 7B4VH2VL2-mFc+Tecentriq combination was comparable to that of the two monospecific antibodies when used alone, while the bi- or multi-specific antibodies of the disclosure had evidently superior anti-tumor effect than the two monospecific antibodies and the 7B34VH2VL2-mFc+tecentriq combination, indicating their excellent in vivo anti-tumor efficacy.

Exemplary sequences in the present application are s-t forth below.

Description/
Sequence/SEQ ID NO.
anti-CD40 antibody 7B4VH2VL2's heavy chain variable region
(SEQ ID NO: 1)
QVQLQESGPGLVKPSDTLSLTCAVSGYSITTNYYWNWIRQPPGKGLEWMGYIKYDGSNN
YNPSLKNRITISVDISKNQFSLKLSSVTAVDTAVYYCARLDYWGQGTLVTVSS
anti-CD40 antibody 7B4VH2VL2's VH-CDR1
(SEQ ID NO: 28)
TNYYWN
anti-CD40 antibody 7B4VH2VL2's VH-CDR2
(SEQ ID NO: 29)
YIKYDGSNNYNPSLKN
anti-CD40 antibody 7B4VH2VL2's VH-CDR3
LDY
anti-CD40 antibody 7B4VH2VL2's light chain variable region
(SEQ ID NO: 2)
DVVLTQSPLSLPVTLGQPASISCRSSQSLENSNGNTFLNWFQQRPGQSPRLLIYKVSNRFS
GVPDRFSGSGSGTDFTLKISRVEAEDVGVYYCLQVTHVPFTFGQGTKLEIK
anti-CD40 antibody 7B4VH2VL2's VL-CDR1
(SEQ ID NO: 30)
RSSQSLENSNGNTFLN
anti-CD40 antibody 7B4VH2VL2's VL-CDR2
(SEQ ID NO: 31)
KYSNRFS
anti-CD40 antibody 7B4VH2VL2's VL-CDR3
(SEQ ID NO: 32)
LQVTHVPFT
anti-PD-L1 antibody 56E5VHSVL4's heavy chain variable region
(SEQ ID NO: 3)
QVQLQESGPGLVKPSQTLSLTCTVSGGSITSDYWNWIRQHPGNKLEYMGYISYTGSTYYN
PSLKSRISITRDTSKNQFSLKLSSVTTADTAVYYCARYRDWDVRAMDYWGQGTTVTVSS
anti-PD-L1 antibody 56E5VHSVL4's VH-CDR1
(SEQ ID NO: 33)
SDYWN
anti-PD-L1 antibody 56E5VHSVL4's VH-CDR2
(SEQ ID NO: 34)
YISYTGSTYYNPSLKS
anti-PD-L1 antibody 56E5VHSVL4's VH-CDR3
(SEQ ID NO: 35)
YRDWDVRAMDY
anti-PD-L1 antibody 56E5VHSVL4's light chain variable region
(SEQ ID NO: 4)
DIVMTQSPDSLAVSLGEKYTMNCKSSQSLLISGNQKNFLTWYQQKPGQPPKLLIYWAST
RESGVPDRESGSGSGTDETLTISSLQAEDLAVYYCQNDFGFPFTFGQGTKVEIK
anti-PD-L1 antibody 56E5VHSVL4's VL-CDR1
(SEQ ID NO: 36)
KSSQSLLISGNQKNFLT
anti-PD-L1 antibody 56E5VHSVL4's VL-CDR2
(SEQ ID NO: 37)
WASTRES
anti-PD-L1 antibody 56E5VHSVL4's VL-CDR3
(SEQ ID NO: 38)
QNDFGFPFT
MBS307-1-1's long chain
(SEQ ID NO: 5)
QVQLQESGPGLVKPSDTLSLTCAVSGYSITTNYYWNWIRQPPGKGLEWMGYIKYDGSNNY
NPSLKNRITISVDTSKNQFSLKLSSVTAVDTAVYYCARLDYWGQGTLVTVSSASTKGPSVF
PLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKD
TLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLWCL
VKGFYPSDIAVEWESNGQPENNYKTTPPVLDSDGSFFLYSKLTVDKSRWQQGNVFSCSVM
HEALHNHYTQKSLSLSPGGGGSGGGGSGGGGSGGGGSGIPPHVQKSVNNDMIVTDNNGA
VKFPQLCKFCDVRFSTCDNQKSCMSNCSITSICEKPQEVCVAVWRKNDENITLETVCHDPK
LPYHDFILEDAASPKCIMKEKKKPGETFFMCSCSSDECNDNIIFSEEYNTSNPD
human κ constnat region
(SEQ ID NO: 6)
RTVAAPSVFIFPPSDEQLKSGTASVYCLLNNFYPREAKVQWKVDNALQSGNSQESVTEQDS
KDSTYSLSSTLILSKADYEKHKVYACEVTHQGLSSPVTKSFNRGEC
MBS307-1-2's long chain
(SEQ ID NO: 7)
QVQLQESGPGLVKPSQTLSLTCTVSGGSITSDYWNWIRQHPGNKLEYMGYISYTGSTYYNP
SLKSRISITRDTSKNQFSLKLSSVTTADTAVYYCARYRDWDVRAMDYWGQGTTVTVSSAS
TKGPSVFPLAPSSKSTSGGTAALGCLVKDYFPEPVTYSWNSGALTSGVHTFPAVLQSSGLY
SLSSVVTVPSSSLGTQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQ
VSLSCAVKGFYPSDIAVEWESNGQPENNYKTTPPVLDSDGSFFLVSKLTVDKSRWQQGNV
FSCSVMHEALHNHYTQKSLSLSPGGGGSGGGGSGGGGSGGGGSGIPPHVGKSVNNDMIVT
DNNGAVKFPQLCKFCDVRESTCDNQKSCMSNCSITSICEKPQEVCVAVWRKNDENITLETV
CHDPKLPYHDFILEDAASPKCIMKEKKKPGETFFMCSCSSDECNDNIIFSEEYNTSNPD
MBS307-2's long chain
(SEQ ID NO: 8)
QVQLQESGPGLVKPSQTLSLTCTVSGGSITSDYWNWIRQHPGNKLEYMGYISYTGSTYYNP
SLKSRISITRDTSKNQPSLKLSSVTTADTAVYYCARYRDWDVRAMDYWGQGTTVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTKPREEQYNSTYRV
VSVLTVLHQDWLNGKEYKCKVSNKALGAPIEKTISKAKGQPREPQVYTLPPSREEMTKNQ
VSLTCLVKGFYPSDIAVEWESNGQPENNYKTTPPVLDSDGSFFLYSKLTVDKSRWQQGNV
FSCSVMHEALHNHYTQKSLSLSPGSGGGGSGGGGSQVQLQESGPGLVKPSDTLSLTCAVS
GYSITTNYYWNWIRQPPGKGLEWMGYIKYDGSNNYNPSLKNRITISVDTSKNQFSLKLSSV
TAVDTAVYYCARLDYWGQGTLVTVSSGGGGSGGGGSGGGGSGGGSDVVLTQSPLSLPVT
LGQPASISCRSSQSLENSNGNTFLNWFQQRPGQSPRLLIYKVSNRFSGVPDRESGSGSGTDF
TLKISRVEAEDVGVYYCLQVTHVPFTFGQGTKLEIK
MBS307-3's long chain
(SEQ ID NO: 9)
QVQLQESGPGLVKPSDTLSLTCAVSGYSITTNYYWNWIRQPPGKGLEWMGYIKYDGSNNY
NPSLKNRITISVDTSKNQFSLKLSSVTAVDTAVYYCARLDYWGQGTLVTVSSASTKGPSVF
PLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKD
TLMISRTPEVTCVVVDVSHEDPEVKFNWYVDGVEVHNAKTKPREEQYNSTYRVVSVLTV
LHQDWLNGKEYKCKVSNKALGAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVM
HEALHNHYTQKSLSLSPGSGGGGSGGGGSQVQLQESGPGLYKPSQTLSLTCTVSGGSITSD
YWNWIRQHPGNKLEYMGYISYTGSTYYNPSLKSRISITRDTSKNQFSLKESSYTTADTAVY
YCARYRDWDVRAMDYWGQGTTVTVSSGGGGSGGGGSGGGGSGGGGSDIVMTQSPDSLA
VSLGEKVTMNCKSSQSLLISGNQKNFLTWYQQKPGQPPKLLIYWASTRESGVPDRFSGSGS
GTDFTLTISSLQAEDEAVYYCQNDFGFPFTFGQGTKLTAK
anti-PD-L1 antibody 3C2VH6VLS's heavy chain variable region
(SEQ ID NO: 10)
QVQLVQSGAEYKKPGASVKVSCKASGYTFTDYHVNWVRQAPGGGLEWIGWIFPGSGRT
FYTDKFKGRVTMTVDTSTSTVYMELSSLRSEDTAVYYCATDYGTSGYGLVYWGQGTTV
TVSS
anti-PD-L1 antibody 3C2VH6VLS's VH-CDR1
(SEQ ID NO: 39)
DYHVN
anti-PD-L1 antibody 3C2VH6VLS's VH-CDR2
(SEQ ID NO: 40)
WIFPGSGRTFYTDKFKG
anti-PD-L1 antibody 3C2VH6VL5's VH-CDR3
(SEQ ID NO: 41)
DYGTSGYGLVY
anti-PD-L1 antibody 3C2VH6VL5's light chain variable region
(SEQ ID NO: 11)
DIQMTQSPSSLSASVGDRYTITCKASDRINNWLAWYQQKPGKAPKLLISGATSLETGVPS
RFSGSGSGTDFTLTISSLQPEDIATYYCQQYWNIPFTFGQGTKVEIK
anti-PD-L1 antibody 3C2VH6VLS's VL-CDR1
(SEQ ID NO: 42)
KASDRINNWLA
anti-PD-L1 antibody 3C2VH6VLS's VL-CDR2
(SEQ ID NO: 43)
GATSLET
anti-PD-L1 antibody 3C2VH6VLS's VL-CDR3
(SEQ ID NO: 44)
QQYWNIPFT
MBS307-6-1 and MBS307-10-1's long chain
(SEQ ID NO: 12)
QVQLQESGPGLVKPSDTLSLTCAVSGYSITTNYYWNWIRQPPGKGLEWMGYIKYDGSNNY
NPSLKNRITISVDTSKNQFSLKLSSVTAVDTAVYYCARLDYWGGGTLVTVSSASTKGPSVF
PLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKD
TLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLWCL
VKGFYPSDIAVEWESNGQPENNYKTTPPVLDSDGSFFLYSKLTVDKSRWQQGNVFSCSVM
HEALHNHYTQKSLSLSPGK
MBS307-6-2 and MBS307-8-2's long chain
(SEQ ID NO: 13)
QVQLQESGPGLVKPSDTLSLTCAVSGYSITTNYYWNWIRQPPGKGLEWMGYIKYDGSNNY
NPSLKNRITISVDTSKNQFSLKLSSVTAVDTAVYYCARLDYWGQGTLVTVSSASTKGPSVF
PLAPSSKSTSGGTAALGCLYKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKD
TLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLSCA
VKGFYPSDIAVEWESNGQPENNYKTTPPVLDSDGSFFLVSKLTVDKSRWQQGNVESCSVM
HEALHNHYTQKSLSLSPGSGGGGSGGGGSQVQLQESGPGLVKPSQTLSLTCTVSGGSITSD
YWNWIRQHPGNKLEYMGYISYTGSTYYNPSLKSRISITRDTSKNQFSLKLSSVTTADTAVY
YCARYRDWDVRAMDYWGQGTTVTYSSGGGGSGGGGSGGGGSGGGGSDIVMTQSPDSLA
VSLGEKVTMNCKSSQSLLISGNQKNFLTWYQQKPGQPPKLLIYWASTRESGVPDRFSGSGS
GTDFTLTISSLQAEDEAVYYCQNDFGFPFTFGQGTKLTAK
MBS307-8-1's long chain
(SEQ ID NO: 14)
QVQLQESGPGLVKPSDTLSLTCAVSGYSITTNYYWNWIRQPPGKGLEWMGYIKYDGSNNY
NPSLKNRITISVDTSKNQFSLKLSSVTAVDTAVYYCARLDYWGQGTLVTVSSASTKGPSVF
PLAPSSKSTSGGTAALGCLYKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKD
TLMISRTPEVTCVVVDVSHEDPEVKFNWYVDGVEVHNAKTKPREEQYNSTYRVVSVLTV
LHQDWINGKEYKCKVSNKALPAPIEKTISKAKGQPREPQVYTLPPSREEMTKNQVSLWCL
VKGFYPSDIAVEWESNGQPENNYKTTPPVLDSDGSFFLYSKLTVDKSRWQQGNVFSCSVM
HEALHNHYTQKSLSLSPGSGGGGSGGGGSQVQLVQSGAEVKKPGASVKYSCKASGYTFT
DYHVNWVRQAPGQGLEWIGWIFPGSGRTFYTDKFKGRVTMTVDISTSTVYMELSSLRSE
DTAVYYCATDYGTSGYGLVYWGQGTTVTVSSGGGGSGGGGSGGGGSGGGGSDIQMTQS
PSSLSASVGDRVTITCKASDRINNWLAWYQQKPGKAPKLLISGATSLETGVPSRFSGSGSGT
DFTLTISSLQPEDEATYYCQQYWNIPFTFGQGTKLTAK
MBS307-9-1's long chain
(SEQ ID NO: 15)
QVQLQESGPGLYKPSDTLSLTCAVSGYSITTNYYWNWIRQPPGKGLEWMGYIKYDGSNNY
NPSLKNRITISVDTSKNQESLKLSSVTAVDTAVYYCARLDYWGQGTLVTVSSASTKGPSVF
PLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLEPPKPKD
TLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLWCL
VKGFYPSDIAVEWESNGQPENNYKTTPPVLDSDGSFFLYSKLTVDKSRWQQGNVFSCSVM
HEALHNHYTQKSLSLSPGGGGGQVQLQESGPGLVKPSQTLSLTCTVSGGSITSDYWNWIR
QHPGNKLEYMGYISYTGSTYYNPSLKSRISITRDTSKNQFSLKLSSVTTADTAVYYCARYR
DWDVRAMDYWGQGTTVTVSSGGGGSGGGGSGGGGSGGGGSDIVMTQSPDSLAVSLGEK
VTMNCKSSQSLLISGNQKNFLTWYQQKPGQPPKLLIYWASTRESGVPDRFSGSGSGTDFTL
TISSLQAEDEAVYYCQNDEGFPFTEGQGTKLTAK
MBS307-9-2, MBS307-10-2 and MBS307-11-2's long chain
(SEQ ID NO: 16)
QVQLQESGPGLVKPSDTLSLTCAVSGYSITTNYYWNWIRQPPGKGLEWMGYIKYDGSNNY
NPSLKNRITISVDTSKNQFSLKLSSVTAVDTAVYYCARLDYWGQGTLVTYSSASTKGPSVF
PLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVELFPPKPKD
TLMISRTPEVTCVVYDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLSCA
VKGFYPSDIAVEWESNGQPENNYKTTPPVLDSDGSFFLVSKLTVDKSRWQQGNVFSCSVM
HEALHNHYTQKSLSLSPGGGGGQVQLVQSGAEVKKPGASVKVSCKASGYTFTDYHVNW
VRQAPGQGLEWIGWIFPGSGRTFYTDKFKGRVTMTVDTSTSTVYMELSSLRSEDTAVYYC
ATDYGTSGYGLVYWGQGTTVTVSSGGGGSGGGGSGGGGSGGGGSDIQMTQSPSSLSASV
GDRVTITCKASDRINNWLAWYQQKPGKAPKLLISGATSLETGVPSRFSGSGSGTDFTLTISS
LQPEDEATYYCQQYWNIPFTFGQGTKLTAK
MBS307-11-1's long chain
(SEQ ID NO: 17)
QVQLQESGPGLVKPSDTLSLTCAVSGYSITTNYYWNWIRQPPGKGLEWMGYIKYDGSNNY
NPSLKNRITISVDTSKNQFSLKLSSVTAVDTAVYYCARLDYWGQGTLVTVSSASTKGPSVF
PLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVELFPPKPKD
TLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLWCL
VKGFYPSDIAVEWESNGQPENNYKTTPPVLDSDGSFPLYSKLTVDKSRWQQGNVFSCSVM
HEALHNHYTQKSLSLSPGGGGGQVQLLESGGGLVQPGGSLRLSCAASGFAFSDYAMTWV
RQAPGKGLEWYSTINSGGQSSSYLDSVKGRFTSSRDNSKNTLYLQMNSLRAEDTAVYYCA
KGVYMPWKAPKGQGTLVTVSS
MBS307-7's long chain
(SEQ ID NO: 18)
QVQLQESGPGLYKPSDTLSLTCAVSGYSITTNYYWNWIRQPPGKGLEWMGYIKYDGSNNY
NPSLKNRITISVDTSKNQFSLKLSSVTAVDTAVYYCARLDYWGQGTLVTVSSASTKGPSVF
PLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVELFPPKPKD
TLMISRTPEVTCVVYDVSHEDPEYKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVM
HEALHNHYTQKSLSLSPGSGGGGSGGGGSQVQLVQSGAEVKKPGASVKYSCKASGYTFT
DYHVNWVRQAPGGGLEWIGWIFPGSGRTFYTDKFKGRVTMTVDISTSTVYMELSSLRSE
DTAVYYCATDYGTSGYGLVYWGQGTTVTVSSGGGGSGGGGGGGGSGGGGSDIQMTQS
PSSLSASVGDRVTITCKASDRINNWLAWYQQKPGKAPKLLISGATSLETGVPSRFSGSGSGT
DFTLTISSLQPEDEATYYCQQYWNIPFTFGQGTKLTAK
linker 1
(SEQ ID NO: 19)
GGGGSGGGGSGGGGSGGGGS
linker 2
(SEQ ID NO: 20)
GGGGSGGGGS
linker 3
(SEQ ID NO: 21)
GSGGGGSGGGGS
linker 4
(SEQ ID NO: 22)
GGGGG
human PD-L1
(SEQ ID NO: 23)
MRIFAVFIFMTYWHLLNAFTVTVPKDLYVVEYGSNMTIECKFPVEKQLDLAALIVYWEME
DKNIIQFVHGEEDLKVQHSSYRQRARLLKDQLSLGNAALQITDVKLQDAGVYRCMISYGG
ADYKRITVKYNAPYNKINQRILVVDPVTSEHELTCQAEGYPKAEVIWTSSDHQVLSGKTTT
TNSKREEKLENVTSTLRINTTTNEIFYCTFRRLDPEENHTAELVIPELPLAHPPNERTHLVILG
AILLCLGVALTFIFRLRKGRMMDVKKCGIQDTNSKKQSDTHLEET
monkey PD-L1
(SEQ ID NO: 24)
MRIFAVFIFTIYWHLLNAFTVTVPKDLYVVEYGSNMTIECKFPVEKQLDLTSLIVYWEMEDK
NIIQFVHGEEDLKVQHSNYRQRAQLLKDQLSLGNAALRITDVKLQDAGVYRCMISYGGAD
YKRITVKVNAPYNKINQRILVVDPVTSEHELTCQAEGYPKAEVIWTSSDHQVLSGKTTTTN
SKREEKLLNVTSTLRINTTANEIFYCIFRRLDPEENHTAELVIPELPLALPPNERTHLVILGAIF
LLLGVALTFIFYLRKGRMMDMKKCGIRVTNSKKQRDTQLEET
human CD40
(SEQ ID NO: 25)
MVRLPLQCVLWGCLLTAVHPEPPTACREKQYLINSQCCSLCQPGQKLVSDCTEFTETECLPC
GESEFLDTWNRETHCHQHKYCDPNLGLRVQQKGTSETDTICTCEEGWHCTSEACESCVLH
RSCSPGFGVKQIATGVSDTICEPCPVGFFSNVSSAFEKCHPWTSCETKDLVVQQAGINKTD
VVCGPQDRERALVVIPIIFGILFAILLVLVFIKKVAKKPTNKAPHPKQEPQEINFPDDLPGSNT
AAPVQETLHGCQPVTQEDGKESRISVQERQ
monkey CD40
(SEQ ID NO: 26)
MVRLPLQCVLWGCLLTAVYPEPPTACREKQYLINSQCCSLCQPGQKLVSDCTEFTETECLPC
SESEFLDTWNRETRCHQHKYCDPNLGLRVQQKGTSETDTICTCEEGLHCMSESCESCVPHR
SCLPGFGVKQIATGVSDTICEPCPVGFFSNVSSAFEKCRPWTSCETKDLVVQQAGTNKTDV
VCGPQDRQRALVVIPICLGILFVILLLVLVFIKKVAKKPNDKAPHPKQEPQEINELDDLPGSN
PAAPVQETLHGCQPVTQEDGKESRISVQERQ
mouse CD40
(SEQ ID NO: 27)
MVSLPRLCALWGCLLTAVHLGQCVTCSDKQYLHDGQCCDLCQPGSRLTSHCTALEKTQCH
PCDSGEFSAQWNREIRCHQHRHCEPNQGLRVKKEGTAESDTVCTCKEGQHCTSKDCEACA
QHTPCIPGFGYMEMATETTDTVCHPCPVGFFSNQSSLFEKCYPWTSCEDKNLEVLQKGTSQ
TNVICGLKSRMRALLVIPVVMGILITIFGVFLYIKKVVKKPKDNEILPPAARRQDPQEMEDYP
GHNTAAPVQETLHGCQPVTQEDGKESRISVQERQVTDSIALRPLV
IgG1 heavy chain constant region with LALA mutation
(SEQ ID NO: 46)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGGPSVF
LFPPKPKDTLMISRTPEVTCVVVDVSHEDPEVKFNWYVDGVEVHNAKTKPREEQYNSTYR
VVSVLTVLHQDWLNGKEYKCKKALGAPIEKTISKAKGQPREPQVCTLPPSREEMTKN
QVSLSCAVKGFYPSDIAVEWESNGQPENNYKTTPPVLDSDGSFELVSKLTVDKSRWQQGNV
FSCSVMHEALHNHYTQKSLSLSP
IgG1 heavy chain constant region with knob and LALA mutations
(SEQ ID NO: 47)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGGPSVF
LFPPKPKDTLMISRIPEVTCVVVDVSHEDPEVKFNWYVDGVEVHNAKTKPREEQYNSTYR
VVSVLTVLHQDWINGKEYKCKVSNKALPAPIEKTISKAKGQPREPQVYTIPPSREEMTKN
QVSLWCLVKGFYPSDIAVEWESNGQPENNYKTTPPVLDSDGSFFLYSKLTVDKSRWQQGN
VFSCSVMHEALHNHYTQKSLSLSP
IgG1 heavy chain constant region with hole and LALA mutations
(SEQ ID NO: 48)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGGPSVF
LFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKN
QVSLSCAVKGFYPSDIAVEWESNGQPENNYKTTPPVLDSDGSFFLVSKLTVDKSRWQQGNV
FSCSVMHEALHNHYTQKSLSLSP
anti-PDLI nanobody's CDR1
(SEQ ID NO: 49)
DYAMT
anti-PDLI nanobody's CDR2
(SEQ ID NO: 50)
TINSGGQSSSYLDSVKG
anti-PDLI nanobody's CDR3
(SEQ ID NO: 51)
GVYMPWKAP
anti-PDLI nanobody
(SEQ ID NO: 52)
QVQLLESGGGLVQPGGSLRLSCAASGFAFSDYAMTWVRQAPGKGLEWVSTINSGGQSSSY
LDSVKGRFTSSRDNSKNTLYLQMNSLRAEDTAVYYCAKGVYMPWKAPKGQGTLVTVSS
MBS307-1-1, MBS307-3, MBS307-6, MBS307-7, MBS307-8, MBS307-9, MBS307-10
and MBS307-11's short chain
(SEQ ID NO: 53)
DVVLTQSPLSLPVTLGQPASISCRSSQSLENSNGNTFLNWFQQRPGQSPRLLIYKVSNRFSGV
PDRFSGSGSGTDFTLKISRVEAEDVGVYYCLQVTHVPFTPGQGTKLEIKRTVAAPSVFIFPPS
DEQLKSGTASVVCLLNNFYPREAKYQWKVDNALQSGNSQESVTEQDSKDSTYSLSSTLTL
SKADYEKHKVYACEVTHQGLSSPVTKSENRGEC
mouse κ light chain constant region
(SEQ ID NO: 54)
RADAAPTVSIFPPSSEQLTSGGASVVCFLNNFYPKDINVKWKIDGSERQNGVLNSWTDQDS
KDSTYSMSSTLILIKDEYERHNSYTCEATHKTSTSPIVKSFNRNEC
mouse IgG1 heavy chain constant region
(SEQ ID NO: 45)
AKTTPPSVYPLAPGSAAQTNSMVTLGCLVKGYFPEPVTVTWNSGSLSSGVHTFPAVLQSDL
YTLSSSVTVPSSTWPSETVTCNVAHPASSTKVDKKIVPRDCGCKPCICTVPEVSSVFIFPPKP
KDVLTITLTPKVTCVVVDISKDDPEVQFSWFVDDVEVHTAQTQPREEQFNSTFRSVSELPIM
HQDWLNGKEFKCRVNSAAFPAPIEKTISKTKGRPKAPQVYTIPPPKEQMAKDKVSLTCMIT
DFFPEDITVEWQWNGQPAENYKNTQPIMDIDGSYFVYSKLNVQKSNWEAGNTFTCSVLH
EGLHNHHTEKSLSHSPGK

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A multi-specific antibody, comprising
i) a first polypeptide chain, comprising, from N terminus to C terminus, an anti-CD40 heavy chain variable region, a heavy chain constant region, and a first binding domain capable of specifically binding PD-L1 and antagonizing PD-1 signaling pathway;
ii) a second polypeptide chain, comprising an anti-CD4) light chain variable region:
iii) a third polypeptide chain, comprising, from N terminus to C terminus, an anti-CD40 heavy chain variable region, and a heavy chain constant region; or comprising, from N terminus to C terminus, an anti-CD40 heavy chain variable region, a heavy chain constant region, and a second binding domain capable of specifically binding PD-L1 and antagonizing PD-1 signaling pathway; and
iv) a fourth polypeptide chain, comprising an anti-CD40 light chain variable region, wherein the anti-CD40 heavy chain variable region in the first polypeptide chain and the anti-CD40 light chain variable region in the second polypeptide chain associate to form a CD40 binding domain capable of specifically binding CD40 and agonizing CD40 signaling pathway, the anti-CD40 heavy chain variable region in the third polypeptide chain and the anti-CD40 light chain variable region in the fourth polypeptide chain associate to form a CD40 binding domain capable of specifically binding CD40 and agonizing CD40 signaling pathway, the heavy chain constant region in the first polypeptide chain and the heavy chain constant region in the third polypeptide chain are associated together.

2. The multi-specific antibody of claim 1, wherein the third polypeptide chain comprise the second binding domain, and the first binding domain and the second binding domain are capable of binding to PD-L1 on the same or different epitopes.

3. The multi-specific antibody of claim 1, wherein the first binding domain is a single-chain variable fragment (scFv) or a nanobody, the second binding domain is a single-chain variable fragment (scFv) or a nanobody.

4. The multi-specific antibody of claim 1, wherein the third polypeptide chain comprises the second binding domain, and the first binding domain and the second binding domain are a single-chain variable fragment and a nanobody, respectively, are both nanobodies, or are both single-chain variable fragments.

5. The multi-specific antibody of claim 4, wherein the single-chain variable fragment comprises a VH-CDR1, a VH-CDR2, a VH-CDR3, a VL-CDR1, a VL-CDR2 and a VL-CDR3 comprising the amino acid sequences of i) SEQ ID NOs: 33, 34, 35, 36, 37 and 38, respectively; or ii) SEQ ID NOs: 39, 40, 41, 42, 43 and 44, respectively, and/or
the nanobody comprises a CDR1, a CDR2 and a CDR3 comprising the amino acid sequences of SEQ ID NOs: 49, 50 and 51, respectively.

6. The multi-specific antibody of claim 5, wherein the single-chain variable fragment comprises a heavy chain variable region and a light chain variable region comprising amino acid sequences having at least 85%, 86%, 87%, 88%, 89%, 90%, 91%, 92%, 93%, 94%, 95%, 96%, 97%, 98%, 99% or 100% sequence identity to i) SEQ ID NOs: 3 and 4, respectively; or
ii) SEQ ID NOs: 10 and 11, respectively, and/or
the nanobody comprises an amino acid sequence having at least 85%, 86%, 87%, 88%, 89%, 90%, 91%, 92%, 93%, 94%, 95%, 96%, 97%, 98%, 99% or 100% sequence identity to SEQ ID NO: 52.

7. The multi-specific antibody of claim 1, wherein the heavy chain constant regions in the first polypeptide chain and the third polypeptide chain are with weak or no FcR binding affinity.

8. The multi-specific antibody of claim 1, wherein the anti-CD40 heavy chain variable region in the first polypeptide chain comprises a VH-CDR1 and a VH-CDR2 comprising amino acid sequences of SEQ ID NOs: 28 and 29, respectively, and a VH-CDR3 comprising the amino acid sequence of ‘LDY’, the anti-CD40 light chain variable region in the second polypeptide chain comprises a VL-CDR1, a VL-CDR2 and a VL-CDR3 comprising the amino acid sequences of SEQ ID NOs: 30, 31 and 32, respectively; and/or
the anti-CD40 heavy chain variable region in the third polypeptide chain comprises a VH-CDR1 and a VH-CDR2 comprising amino acid sequences of SEQ ID NOs: 28 and 29, respectively, and a VH-CDR3 comprising the amino acid sequence of ‘LDY’, the anti-CD40 light chain variable region in the fourth polypeptide chain comprises a VL-CDR1, a VL-CDR2 and a VL-CDR3 comprising the amino acid sequences of SEQ ID NOs: 30, 31 and 32, respectively.

9. The multi-specific antibody of claim 8, wherein the anti-CD40 heavy chain variable region and the anti-CD40 light chain variable region comprise the amino acid sequences having at least 85%, 86%, 87%, 88%, 89%, 90%, 91%, 92%, 93%, 94%, 95%, 96%, 97%, 98%, 99% or 100% sequence identity to SEQ ID NOs: 1 and 2, respectively.

10. The multi-specific antibody of claim 1, wherein the heavy chain constant region in the first polypeptide chain is linked via a linker to the first binding domain, and/or the heavy chain constant region in the third polypeptide chain is linked via a linker to the second binding domain.

11. The multi-specific antibody of claim 10, wherein the linker comprises the amino acid sequence of SEQ ID NOs: 19, 20, 21 or 22.

12. The multi-specific antibody of claim 1, wherein
i) the first polypeptide chain comprises, from N terminus to C terminus, the anti-CD40 heavy chain variable region, the heavy chain constant region, a linker, an anti-PD-L1 heavy chain variable region, a linker, and an anti-PD-L1 light chain variable region; or comprises, from N terminus to C terminus, the anti-CD40 heavy chain variable region, the heavy chain constant region, a linker, and an anti-PD-L1 nanobody;
ii) the second polypeptide chain comprises, from N terminus to C terminus, the anti-CD40 light chain variable region and a light chain constant region:
iii) the third polypeptide chain comprises, from N terminus to C terminus, the anti-CD40 heavy chain variable region, the heavy chain constant region, a linker, an anti-PD-L1 heavy chain variable region, a linker, and an anti-PD-L1 light chain variable region; or comprises, from N terminus to C terminus, the anti-CD40 heavy chain variable region, the heavy chain constant region, a linker, and an anti-PD-L1 nanobody; and
iv) the fourth polypeptide chain comprises, from N terminus to C terminus, the anti-CD40 light chain variable region and a light chain constant region,
wherein the anti-PD-L1 heavy chain variable region and the anti-PD-L1 light chain variable region in the first polypeptide chain associate to form the first binding domain, or the anti-PD-L1 nanobody in the first polypeptide chain forms the first binding domain; the anti-PD-L1 heavy chain variable region and the anti-PD-L1 light chain variable region in the third polypeptide chain associate to form the second binding domain, or the anti-PD-L1 nanobody in the third polypeptide chain forms the second binding domain,
wherein the first binding domain and the second binding domain are capable of binding to PD-L1 on the same or different epitopes.

13. The multi-specific antibody of claim 12, wherein the first, second, third and fourth polypeptide chains comprise amino acid sequences having at least 85%, 86%, 87%, 88%, 89%, 90%, 91%, 92%, 93%, 94%, 95%, 96%, 97%, 98%, 99% or 100% sequence identity to i) SEQ ID NOs: 17, 53, 16 and 53, respectively; ii) SEQ ID NOs: 9, 53, 9 and 53, respectively; iii) SEQ ID NOs: 12, 53, 13 and 53, respectively; iv) SEQ ID NOs: 18, 53, 18 and 53, respectively; v) SEQ ID NOs: 14, 53, 13 and 53, respectively; vi) SEQ ID NOs: 15, 53, 16 and 53, respectively; or vii) SEQ ID NOs: 12, 53, 16 and 53, respectively.

14. A nucleic acid molecule encoding the multi-specific antibody of claim 1.

15. An expression vector comprising the nucleic acid molecule of claim 14.

16. A host cell comprising the expression vector of claim 15.

17. A pharmaceutical composition comprising the multi-specific antibody of claim 1, and a pharmaceutically acceptable carrier.

18. A method for treating a tumor or an infectious disease in a subject in need thereof, comprising administering to the subject the pharmaceutical composition of claim 17.

19. The method of claim 18, wherein the tumor is melanoma, lung cancer, renal cell carcinoma, Hodgkin lymphoma, bladder cancer, head and neck cancer, neuroendocrine cancer, mantle cell lymphoma, B cell lymphoma, follicular lymphoma, multiple myeloma, rectal adenocarcinoma, pancreatic cancer, colorectal cancer, gastric cancer, prostate cancer, kidney cancer, ovarian cancer, cervical cancer, breast cancer, or nasopharyngeal cancer.

20. The method of claim 18, wherein the infectious disease is chronic infection of hepatitis B virus, hepatitis C virus, human immunodeficiency virus, or simian immunodeficiency virus.


SEQUENCE LISTING (54 sequences)

SEQ ID NO: 1 - protein, 112 aa, synthetic construct
QVQLQESGPGLVKPSDTLSLTCAVSGYSITTNYYWNWIRQPPGKGLEWMGYIKYDGSNNYNPSLKNRITISVDTSKNQFSLKLSSVTAVDTAVYYCARLDYWGQGTLVTVSS

SEQ ID NO: 2 - protein, 112 aa, synthetic construct
DVVLTQSPLSLPVTLGQPASISCRSSQSLENSNGNTFLNWFQQRPGQSPRLLIYKVSNRFSGVPDRFSGSGSGTDFTLKISRVEAEDVGVYYCLQVTHVPFTFGQGTKLEIK

SEQ ID NO: 3 - protein, 119 aa, synthetic construct
QVQLQESGPGLVKPSQTLSLTCTVSGGSITSDYWNWIRQHPGNKLEYMGYISYTGSTYYNPSLKSRISITRDTSKNQFSLKLSSVTTADTAVYYCARYRDWDVRAMDYWGQGTTVTVSS

SEQ ID NO: 4 - protein, 113 aa, synthetic construct
DIVMTQSPDSLAVSLGEKVTMNCKSSQSLLISGNQKNFLTWYQQKPGQPPKLLIYWASTRESGVPDRFSGSGSGTDFTLTISSLQAEDLAVYYCQNDFGFPFTFGQGTKVEIK

SEQ ID NO: 5 - protein, 597 aa, synthetic construct
QVQLQESGPGLVKPSDTLSLTCAVSGYSITTNYYWNWIRQPPGKGLEWMGYIKYDGSNNYNPSLKNRITISVDTSKNQFSLKLSSVTAVDTAVYYCARLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 6 - protein, 107 aa, synthetic construct [sequence omitted]

SEQ ID NO: 7 - protein, 604 aa, synthetic construct
QVQLQESGPGLVKPSQTLSLTCTVSGGSITSDYWNWIRQHPGNKLEYMGYISYTGSTYYNPSLKSRISITRDTSKNQFSLKLSSVTTADTAVYYCARYRDWDVRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 8 - protein, 702 aa, synthetic construct
QVQLQESGPGLVKPSQTLSLTCTVSGGSITSDYWNWIRQHPGNKLEYMGYISYTGSTYYNPSLKSRISITRDTSKNQFSLKLSSVTTADTAVYYCARYRDWDVRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSQVQLQESGPGLVKPSDTLSLTCAVSGYSITTNYYWNWIRQPPGKGLEWMGYIKYDGSNNYNPSLKNRITISVDTSKNQFSLKLSSVTAVDTAVYYCARLDYWGQGTLVTVSSGGGGSGGGGSGGGGSGGGSDVVLTQSPLSLPVTLGQPASISCRSSQSLENSNGNTFLNWFQQRPGQSPRLLIYKVSNRFSGVPDRFSGSGSGTDFTLKISRVEAEDVGVYYCLQVTHVPFTFGQGTKLEIK

SEQ ID NO: 9 - protein, 704 aa, synthetic construct
QVQLQESGPGLVKPSDTLSLTCAVSGYSITTNYYWNWIRQPPGKGLEWMGYIKYDGSNNYNPSLKNRITISVDTSKNQFSLKLSSVTAVDTAVYYCARLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSQVQLQESGPGLVKPSQTLSLTCTVSGGSITSDYWNWIRQHPGNKLEYMGYISYTGSTYYNPSLKSRISITRDTSKNQFSLKLSSVTTADTAVYYCARYRDWDVRAMDYWGQGTTVTVSSGGGGSGGGGSGGGGSGGGGSDIVMTQSPDSLAVSLGEKVTMNCKSSQSLLISGNQKNFLTWYQQKPGQPPKLLIYWASTRESGVPDRFSGSGSGTDFTLTISSLQAEDEAVYYCQNDFGFPFTFGQGTKLTAK

SEQ ID NO: 10 - protein, 120 aa, synthetic construct
QVQLVQSGAEVKKPGASVKVSCKASGYTFTDYHVNWVRQAPGQGLEWIGWIFPGSGRTFYTDKFKGRVTMTVDTSTSTVYMELSSLRSEDTAVYYCATDYGTSGYGLVYWGQGTTVTVSS

SEQ ID NO: 11 - protein, 107 aa, synthetic construct
DIQMTQSPSSLSASVGDRVTITCKASDRINNWLAWYQQKPGKAPKLLISGATSLETGVPSRFSGSGSGTDFTLTISSLQPEDIATYYCQQYWNIPFTFGQGTKVEIK

SEQ ID NO: 12 - protein, 442 aa, synthetic construct
QVQLQESGPGLVKPSDTLSLTCAVSGYSITTNYYWNWIRQPPGKGLEWMGYIKYDGSNNYNPSLKNRITISVDTSKNQFSLKLSSVTAVDTAVYYCARLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 704 aa, synthetic construct
QVQLQESGPGLVKPSDTLSLTCAVSGYSITTNYYWNWIRQPPGKGLEWMGYIKYDGSNNYNPSLKNRITISVDTSKNQFSLKLSSVTAVDTAVYYCARLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSQVQLQESGPGLVKPSQTLSLTCTVSGGSITSDYWNWIRQHPGNKLEYMGYISYTGSTYYNPSLKSRISITRDTSKNQFSLKLSSVTTADTAVYYCARYRDWDVRAMDYWGQGTTVTVSSGGGGSGGGGSGGGGSGGGGSDIVMTQSPDSLAVSLGEKVTMNCKSSQSLLISGNQKNFLTWYQQKPGQPPKLLIYWASTRESGVPDRFSGSGSGTDFTLTISSLQAEDEAVYYCQNDFGFPFTFGQGTKLTAK

SEQ ID NO: 14 - protein, 699 aa, synthetic construct
QVQLQESGPGLVKPSDTLSLTCAVSGYSITTNYYWNWIRQPPGKGLEWMGYIKYDGSNNYNPSLKNRITISVDTSKNQFSLKLSSVTAVDTAVYYCARLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGSGGGGSGGGGSQVQLVQSGAEVKKPGASVKVSCKASGYTFTDYHVNWVRQAPGQGLEWIGWIFPGSGRTFYTDKFKGRVTMTVDTSTSTVYMELSSLRSEDTAVYYCATDYGTSGYGLVYWGQGTTVTVSSGGGGSGGGGSGGGGSGGGGSDIQMTQSPSSLSASVGDRVTITCKASDRINNWLAWYQQKPGKAPKLLISGATSLETGVPSRFSGSGSGTDFTLTISSLQPEDEATYYCQQYWNIPFTFGQGTKLTAK

SEQ ID NO: 15 - protein, 697 aa, synthetic construct
QVQLQESGPGLVKPSDTLSLTCAVSGYSITTNYYWNWIRQPPGKGLEWMGYIKYDGSNNYNPSLKNRITISVDTSKNQFSLKLSSVTAVDTAVYYCARLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGQVQLQESGPGLVKPSQTLSLTCTVSGGSITSDYWNWIRQHPGNKLEYMGYISYTGSTYYNPSLKSRISITRDTSKNQFSLKLSSVTTADTAVYYCARYRDWDVRAMDYWGQGTTVTVSSGGGGSGGGGSGGGGSGGGGSDIVMTQSPDSLAVSLGEKVTMNCKSSQSLLISGNQKNFLTWYQQKPGQPPKLLIYWASTRESGVPDRFSGSGSGTDFTLTISSLQAEDEAVYYCQNDFGFPFTFGQGTKLTAK

SEQ ID NO: 16 - protein, 692 aa, synthetic construct
QVQLQESGPGLVKPSDTLSLTCAVSGYSITTNYYWNWIRQPPGKGLEWMGYIKYDGSNNYNPSLKNRITISVDTSKNQFSLKLSSVTAVDTAVYYCARLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGQVQLVQSGAEVKKPGASVKVSCKASGYTFTDYHVNWVRQAPGQGLEWIGWIFPGSGRTFYTDKFKGRVTMTVDTSTSTVYMELSSLRSEDTAVYYCATDYGTSGYGLVYWGQGTTVTVSSGGGGSGGGGSGGGGSGGGGSDIQMTQSPSSLSASVGDRVTITCKASDRINNWLAWYQQKPGKAPKLLISGATSLETGVPSRFSGSGSGTDFTLTISSLQPEDEATYYCQQYWNIPFTFGQGTKLTAK

SEQ ID NO: 17 - protein, 563 aa, synthetic construct
QVQLQESGPGLVKPSDTLSLTCAVSGYSITTNYYWNWIRQPPGKGLEWMGYIKYDGSNNYNPSLKNRITISVDTSKNQFSLKLSSVTAVDTAVYYCARLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGQVQLLESGGGLVQPGGSLRLSCAASGFAFSDYAMTWVRQAPGKGLEWVSTINSGGQSSSYLDSVKGRFTSSRDNSKNTLYLQMNSLRAEDTAVYYCAKGVYMPWKAPKGQGTLVTVSS

SEQ ID NO: 18 - protein, 699 aa, synthetic construct
QVQLQESGPGLVKPSDTLSLTCAVSGYSITTNYYWNWIRQPPGKGLEWMGYIKYDGSNNYNPSLKNRITISVDTSKNQFSLKLSSVTAVDTAVYYCARLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSQVQLVQSGAEVKKPGASVKVSCKASGYTFTDYHVNWVRQAPGQGLEWIGWIFPGSGRTFYTDKFKGRVTMTVDTSTSTVYMELSSLRSEDTAVYYCATDYGTSGYGLVYWGQGTTVTVSSGGGGSGGGGSGGGGSGGGGSDIQMTQSPSSLSASVGDRVTITCKASDRINNWLAWYQQKPGKAPKLLISGATSLETGVPSRFSGSGSGTDFTLTISSLQPEDEATYYCQQYWNIPFTFGQGTKLTAK

SEQ ID NO: 19 - protein, 20 aa, synthetic construct
GGGGSGGGGSGGGGSGGGGS

SEQ ID NO: 20 - protein, 10 aa, synthetic construct
GGGGSGGGGS

SEQ ID NO: 21 - protein, 12 aa, synthetic construct
GSGGGGSGGGGS

SEQ ID NO: 22 - protein, 5 aa, synthetic construct
GGGGG

SEQ ID NO: 23 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 24 - protein, 290 aa, Macaca fascicularis
MRIFAVFIFTIYWHLLNAFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERTHLVILGAIFLLLGVALTFIFYLRKGRMMDMKKCGIRVTNSKKQRDTQLEET

SEQ ID NO: 25 - protein, 277 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 26 - protein, 278 aa, Macaca mulatta
MVRLPLQCVLWGCLLTAVYPEPPTACREKQYLINSQCCSLCQPGQKLVSDCTEFTETECLPCSESEFLDTWNRETRCHQHKYCDPNLGLRVQQKGTSETDTICTCEEGLHCMSESCESCVPHRSCLPGFGVKQIATGVSDTICEPCPVGFFSNVSSAFEKCRPWTSCETKDLVVQQAGTNKTDVVCGPQDRQRALVVIPICLGILFVILLLVLVFIKKVAKKPNDKAPHPKQEPQEINFLDDLPGSNPAAPVQETLHGCQPVTQEDGKESRISVQERQ

SEQ ID NO: 27 - protein, 289 aa, Mus musculus
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 28 - protein, 6 aa, synthetic construct
TNYYWN

SEQ ID NO: 29 - protein, 16 aa, synthetic construct
YIKYDGSNNYNPSLKN

SEQ ID NO: 30 - protein, 16 aa, synthetic construct
RSSQSLENSNGNTFLN

SEQ ID NO: 31 - protein, 7 aa, synthetic construct
KVSNRFS

SEQ ID NO: 32 - protein, 9 aa, synthetic construct
LQVTHVPFT

SEQ ID NO: 33 - protein, 5 aa, synthetic construct
SDYWN

SEQ ID NO: 34 - protein, 16 aa, synthetic construct
YISYTGSTYYNPSLKS

SEQ ID NO: 35 - protein, 11 aa, synthetic construct
YRDWDVRAMDY

SEQ ID NO: 36 - protein, 17 aa, synthetic construct
KSSQSLLISGNQKNFLT

SEQ ID NO: 37 - protein, 7 aa, synthetic construct
WASTRES

SEQ ID NO: 38 - protein, 9 aa, synthetic construct
QNDFGFPFT

SEQ ID NO: 39 - protein, 5 aa, synthetic construct
DYHVN

SEQ ID NO: 40 - protein, 17 aa, synthetic construct
WIFPGSGRTFYTDKFKG

SEQ ID NO: 41 - protein, 11 aa, synthetic construct
DYGTSGYGLVY

SEQ ID NO: 42 - protein, 11 aa, synthetic construct
KASDRINNWLA

SEQ ID NO: 43 - protein, 7 aa, synthetic construct
GATSLET

SEQ ID NO: 44 - protein, 9 aa, synthetic construct
QQYWNIPFT

SEQ ID NO: 45 - protein, 324 aa, synthetic construct
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 46 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 47 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 48 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 49 - protein, 5 aa, synthetic construct
DYAMT

SEQ ID NO: 50 - protein, 17 aa, synthetic construct
TINSGGQSSSYLDSVKG

SEQ ID NO: 51 - protein, 9 aa, synthetic construct
GVYMPWKAP

SEQ ID NO: 52 - protein, 118 aa, synthetic construct
QVQLLESGGGLVQPGGSLRLSCAASGFAFSDYAMTWVRQAPGKGLEWVSTINSGGQSSSYLDSVKGRFTSSRDNSKNTLYLQMNSLRAEDTAVYYCAKGVYMPWKAPKGQGTLVTVSS

SEQ ID NO: 53 - protein, 219 aa, synthetic construct
DVVLTQSPLSLPVTLGQPASISCRSSQSLENSNGNTFLNWFQQRPGQSPRLLIYKVSNRFSGVPDRFSGSGSGTDFTLKISRVEAEDVGVYYCLQVTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC